(19) United States
(12) Patent Application Publication
(10) Pub. No.: US 2024/0122975 A1
(43) Pub. Date: Apr. 18, 2024

(54) IN VITRO METHODS AND COMPOSITIONS FOR ENHANCING THE ACTIVATION OF DENDRITIC CELLS AND T CELLS, AND FOR INDUCING A TH-1 IMMUNE RESPONSE

(71) Applicant: Northwest Biotherapeutics, Inc., Bethesda, MD (US)

(72) Inventors: Marnix Leo Bosch, 's-Gravenhage (NL); Linda F. Powers, Bethesda, MD (US)

(73) Assignee: Northwest Biotherapeutics, Inc., Bethesda, MD (US)

(21) Appl. No.: 17/766,904

(22) PCT Filed: Oct. 7, 2020
(86) PCT No.: PCT/US2020/054621

§ 371 Date: Apr. 6, 2022

Related U.S. Application Data
(60) Provisional application No. 62/912,005, filed on Oct. 7, 2019.

(51) Int. Cl. A61K 35/15; A61K 35/17; A61K 39/00; A61K 45/06; A61P 35/00; C12N 5/0784

(52) U.S. Cl. CPC A61K 35/15; A61K 35/17; A61K 39/4615; A61K 45/06; A61P 35/00; C12N 5/0639

(57) ABSTRACT

The present disclosure provides compositions and in vitro or ex vivo methods for obtaining an enhanced antigen specific Th1 immune response. The compositions can comprise activation enhanced dendritic cells or T cells produced in vitro. The methods comprise contacting immature dendritic cells with a maturation agent that comprises a dendritic cell maturation agent, interferon γ and an inflammation-activating lipid which can produced hyperactivated dendritic cells. The method can further comprise contacting the maturing dendritic cells with a predetermined antigen during maturation. An in vitro or ex vivo method is also provided wherein the hyperactive dendritic cells can be used to induce naive T cell activation, where the activated T cells can be formulated for administration to an individual in need of such treatment.

### CROSS-REFERENCE TO RELATED APPLICATION

This application claims the benefit of U.S. Provisional Application No. 62/912,005, filed Oct. 7, 2019, the disclosure of which is incorporated herein in its entirety.

### BACKGROUND

Antigen presenting cells (APC) are important in eliciting an effective immune response. They not only present antigens to T cells with antigen-specific T cell receptors, but also provide the signals necessary for T cell activation. These signals remain incompletely defined but involve a variety of cell surface molecules as well as cytokines or growth factors. The factors necessary for the activation of naïve or unpolarized T cells may be different from those required for the re-activation of memory T cells. The ability of APC to both present antigens and deliver signals for T cell activation is commonly referred to as an accessory cell function. Although monocytes and B cells have been shown to be competent APC, their antigen presenting capacities appear to be limited to their activation of previously sensitized T cells. Hence, they are not capable of directly activating functionally naïve or unprimed T cell populations.

Dendritic cells (DCs) are the professional antigen presenting cells of the immune system that are believed to be capable of activating both naïve and memory T cells. Dendritic cells are increasingly prepared ex vivo for use in immunotherapy, particularly the immunotherapy of cancer. The preparation of dendritic cells with optimal immunostimulatory properties requires an understanding and exploitation of the biology of these cells for ex vivo culture. Various protocols for the culture of these cells have been described, with various advantages ascribed to each protocol. Recent protocols include the use of serum-free media, and the employment of maturation conditions that impart the desired immunostimulatory properties to the cultured cells.

Maturation of dendritic cells is the process that converts immature DCs, which are phenotypically similar to skin Langerhans cells, to mature, antigen presenting cells that can migrate to the lymph nodes. This process results in the loss of the powerful antigen uptake capacity that characterizes the immature dendritic cells, and in the up-regulation in expression of co-stimulatory cell surface molecules and various cytokines.

Many known maturation protocols are based on the in vivo environment that DCs are believed to encounter during or after exposure to antigens. The best example of this approach is the use of monocyte conditioned media (MCM) as a cell culture medium. MCM is generated in vitro by culturing monocytes and used as a source of maturation factors. The major components in MCM responsible for maturation are reported to be the (pro)inflammatory cytokines Interleukin 1 beta (IL-1β), Interleukin 6 (IL-6) and tumor necrosis factor alpha (TNFα). Other maturation factors include prostaglandin E2 (PGE2), poly-dIdC, vasointestinal peptide (VIP), bacterial lipopolysaccharide (LPS), Bacillus Calmette-Guerin (BCG), as well as other mycobacteria or components of mycobacteria, such as specific cell wall constituents.

Enhancement of IL-12 production by dendritic cells has been reported by combining interferon gamma (IFNγ) with certain dendritic cell maturation factors, such as bacterial lipopolysaccharide (LPS), BCG, and CD40L. LPS, BCG and CD40L have a known capacity to induce small amounts of IL-12 during maturation, however. In addition, BCG has been shown to induce substantial amounts of IL-10, thus originally thought to induce a Th2 immune response when used alone. It has now been shown that the addition of IFNγ enhances the production of IL-12 when combined with LPS, BCG or CD40L when added to immature dendritic cells cultured in vitro. Interferon gamma signaling uses the Jak2-Stat1 pathway, which includes tyrosine phosphorylation of the tyrosine residue at position 701 of Stat1 prior to its migration to the nucleus and the ensuing enhancement of transcription of interferon gamma-responsive genes. Still little is known, however, about signal transduction pathways in human monocyte-derived dendritic cells. The mechanism for IFNγ action in these cells has not been fully established.

Recently, it has been shown that hyperactive DCs secrete inflammatory cytokines from the cytosol (e.g., IL-1β) and biosynthetic pathways (e.g., TNFα) while maintaining viability. These attributes have been achieved by a variety of microbial or host-derived products, including a mixture of oxidized phospholipids, such as oxidized 1-palmitoyl-2-arachidonyl-sn-glycero-3-phosphorylcholine (oxPAPC), its substituents and derivatives.

The Interleukin-1 (IL-1) family of cytokines, including IL-1 and IL-18, play a role in T cell biology generally, and particularly in memory T cell generation and effector function of CD8⁺ T cells. In addition, IL-1 activates intrinsic IL-1 receptor (IL-1R) signaling in pre-committed T helper (TH) cell lineages, and acts as a signal for increased T effector cytokine production. IL-1 usage has been suggested as an adjuvant in weak or inefficient vaccines, to increase vaccination protection activity. Accordingly, inflammation-activators such as oxidized phospholipids have been examined as inducers of hyperactive dendritic cells in vivo capable of enhancing hyperactivation of dendritic cells and inducing an improved antigen specific immune response when the oxidized lipids are administered to a patient with an immunogen.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present invention provides methods and compositions for inducing the in vitro or ex vivo maturation of immature dendritic cells (DC) to produce hyperactive mature dendritic cells, with a combination of agents that simultaneously provide broad immune stimulation (i.e., a dendritic cell maturation agent with or without Interferon γ in combination with an inflammation-activating lipid), and for priming those cells for an enhanced immune response, including an enhance T cell response, an enhanced Th1 response, and/or antigen-specific cytotoxic T cell response.

In one aspect, a method is provided for producing a mature hyperactive dendritic cell population, including providing immature dendritic cells; and contacting the immature dendritic cells in vitro or ex vivo with an effective concentration of dendritic cell maturation agent with or without Interferon γ and an inflammation-activating lipid under culture conditions suitable for maturation of the immature dendritic cells to form a hyperactive mature dendritic cell population. The mature hyperactive dendritic cell population produces an increased level of IL-1, in particular, IL-1β, an increase in effector and memory T cell generation, and an increase in terminal differentiation and activation of CD4⁺ and CD8⁺ T cells than induced by a mature dendritic cell population contacted in vitro or ex vivo with only the dendritic cell maturation agent with or without Interferon γ and without the inflammation-activating lipid. Similarly, the hyperactivated DCs produce greater amounts of Th-1 stimulating cytokines and smaller amounts of Th-2 stimulating cytokines than induced by a mature dendritic cell population contacted in vitro or ex vivo with only the dendritic cell maturation agent in combination with or without Interferon γ and without the inflammation-activating lipid.

The immature dendritic cells can be contacted in vitro or ex vivo with a predetermined antigen prior to, simultaneously with, or subsequent to contact with the dendritic cell maturation agent with or without Interferon γ and the inflammation-activating lipid. Where the predetermined antigen is contacted with the dendritic cells after maturation, or when antigen uptake has been substantially reduced, the antigen can be internalized by known means such as, for example, osmotic loading.

The predetermined antigen can be, for example, a tumor specific antigen, a tumor associated antigen, a viral antigen, a bacterial antigen, tumor cells, bacterial cells, transformed or transfected cells expressing a recombinant antigen, such as a tumor associated tumor antigen, or tumor specific antigen, a cell lysate, such as a bacterial or a tumor cell lysate, a bacterial or tumor cell membrane preparation, a recombinantly produced tumor associated or tumor specific antigen, a peptide antigen (e.g., a synthetic tumor associated, or tumor specific peptide associated or tumor specific antigen), or an isolated antigen, for example, an isolated bacterial, viral, tumor associated or tumor specific antigen.

In certain embodiments, the method can optionally further include isolating or enriching for monocytic dendritic cell precursors; and culturing the precursors in vitro or ex vivo in the presence of a dendritic cell differentiation agent to form a population of immature dendritic cells. Suitable dendritic cell differentiation agents include, for example, GM-CSF, or a combination of GM-CSF and Interleukin 4 (IL-4), Interleukin 7 (IL-7), Interleukin 13 (IL-13), or Interleukin 15 (IL-15). The monocytic dendritic cell precursors can be isolated from a human subject.

In another aspect, an in vitro or ex vivo method for producing a mature hyperactive dendritic cell population is provided. The method generally includes providing immature dendritic cells; and contacting the immature dendritic cells in vitro or ex vivo with an effective amount of a dendritic cell maturation agent with or without Interferon γ and an inflammation-activating lipid under culture conditions suitable for maturation of the immature dendritic cells to form the mature hyperactive dendritic cell population. The resulting mature hyperactive dendritic cell population produces an enhanced immune response. The enhanced immune response can be an enhanced type 1 immune response. The immature dendritic cells can be contacted in vitro or ex vivo with a predetermined antigen prior to, simultaneously with, or subsequent contacting the immature dendritic cells with the dendritic cell maturation agent in combination with or without Interferon γ and the inflammation-activating lipid. When the antigen is contacted with the dendritic cell after full maturation or when antigen uptake is substantially reduced, antigen can be transported into the dendritic cell by known means, such as, for example, osmotic loading.

The predetermined antigen can be, for example, a tumor specific antigen, a tumor associated antigen, a viral antigen, a bacterial antigen, tumor cells, bacterial cells, a transfected or transformed cell expressing a recombinant antigen, such as a tumor associated or tumor specific antigen, a bacterial antigen, or a viral antigen, a cell lysate, such as a tumor cell or bacterial cell lysate, a membrane preparation, such as a tumor or bacterial cell membrane preparation, a recombinantly produced antigen, a peptide antigen (i.e., a synthetic peptide), or an isolated antigen, wherein the antigen is a bacterial, viral, tumor associated, or tumor specific antigen.

In certain embodiments, the in vitro or ex vivo method can optionally further include isolating or providing a cell population enriched for monocytic dendritic cell precursors; and culturing the precursors in vitro or ex vivo in the presence of a dendritic cell differentiation agent to form the immature dendritic cells. Suitable dendritic cell differentiation agents include, for example, GM-CSF, or a combination of GM-CSF and IL-4, IL-13 or IL-15. The monocytic dendritic cell precursors can be isolated from a human subject.

In still another aspect, compositions for activating T cells are provided. The compositions can include a dendritic cell population matured in vitro or ex vivo with an effective concentration of a dendritic cell maturation agent with or without Interferon γ (IFNγ) and the inflammation-activating lipid under suitable conditions for maturation; and a predetermined antigen. The dendritic cell population can produce an increased level of IL-1, in particular, IL-1β, an enhanced immune response, and preferably an increase in a Th1 response, and more preferably an increase in effector and memory T cell generation, and an increase in terminal differentiation and/or activation of CD4⁺ and CD8⁺ T cells than induced by a mature dendritic cell population contacted in vitro or ex vivo with only the dendritic cell maturation agent with or without Interferon γ and without the inflammation-activating lipid.

In another aspect, a composition is provided that comprises isolated, immature dendritic cell population. The cell population includes immature monocytic dendritic cells, and an effective concentration of dendritic cell maturation agent with or without Interferon γ and an inflammation-activating lipid to induce maturation of the immature dendritic cells. The resulting mature hyperactive dendritic cells produces an increased level of IL-1, in particular, IL-1β, an increase in effector and memory T cell generation, and an increase in terminal differentiation and/or of CD4⁺ and CD8⁺ T cells than induced by a mature dendritic cell population contacted in vitro or ex vivo with only the dendritic cell maturation agent with or without Interferon γ and without the inflammation-activating lipid. The cell population can optionally include a predetermined antigen and/or isolated T cells, such as naïve T cells. The T cell can optionally be present in a preparation of isolated lymphocytes.

An in vitro or ex vivo method for producing activated T cells is also provided. The method generally includes providing immature dendritic cells; contacting the immature dendritic cells in vitro or e vivo with a predetermined antigen; and contacting the immature dendritic cells with an effective concentration of dendritic cell maturation agent with or without Interferon γ and an inflammation-activating lipid under culture conditions suitable for maturation of the immature dendritic cells to form mature hyperactive dendritic cells. The mature hyperactive dendritic cells can be contacted with naïve T cells to form activated T cells producing enhanced levels of IFNγ and/or polarized for a type 1 (Th-1) response. Suitable antigens include, for example, a tumor specific antigen, a tumor associated antigen, a viral antigen, a bacterial antigen, tumor cells, bacterial cells, transformed or transfected cells expressing a recombinant antigen, such as a bacterial, viral, tumor associated, or tumor specific antigen, a cell lysate, such as a bacterial or tumor cell lysate, a membrane preparation, such as a bacterial or tumor cell membrane preparation, a recombinantly produced antigen, a peptide antigen (e.g., a synthetic peptide antigen), or an isolated antigen, wherein the antigen is a bacterial, viral, tumor associated or tumor specific antigen.

The immature dendritic cells can be contacted simultaneously with the predetermined antigen, the dendritic cell maturation agent with or without Interferon γ and the inflammation-activating lipid, or the cells can be contacted with the predetermined antigen prior to contacting with the dendritic cell maturation agent with or without Interferon γ and the inflammation-activating lipid. The dendritic cells can also be contacted with the antigen subsequent to full maturation and antigen uptake enhanced using a known method, such as, for example, osmotic loading. In certain embodiments, the in vitro or ex vivo method can further include isolating monocytic dendritic cell precursors; and culturing the precursors in vitro or ex vivo in the presence of a dendritic cell differentiation agent to induce the formation of the immature dendritic cells. Suitable dendritic cell differentiation agents include, for example, GM-CSF, or a combination of GM-CSF and IL-4, IL-13, or IL-15. The monocytic dendritic cell precursors can optionally be isolated from a human subject. In a particular embodiment the immature dendritic cells and T cells are autologous or heterologous to each other.

Isolated mature hyperactive dendritic cells producing more IL-12 are also provided. The mature dendritic cells can be provided by maturing of immature dendritic cells in vitro or ex vivo with a composition comprising effective concentrations of dendritic cell maturation agent with or without Interferon γ and an inflammation-activating lipid under conditions suitable for the maturation of the immature dendritic cells. A predetermined antigen can optionally be included with the isolated, mature dendritic cells. Isolated mature hyperactive dendritic cells loaded with a predetermined antigen are also provided. The dendritic cells can produce more Interleukin 12 (IL-12) and IL-1β than a similar immature dendritic cell population cultured in the presence of the bacteria, the dendritic cell maturation agent with or without Interferon γ and without the inflammation-activating lipid during maturation.

A method for producing a type 1 (Th-1) immune response in an animal is also provided. The method generally includes providing immature dendritic cells; contacting the immature dendritic cells in vitro or ex vivo with an effective amount of dendritic cell maturation agent with or without Interferon γ and an inflammation-activating lipid, and a predetermined antigen under in vitro or ex vivo culture conditions suitable for maturation of the immature dendritic cells to form mature hyperactive dendritic cells. The mature hyperactive dendritic cells can either be administered to an animal or can be contacted in vitro or ex vivo with naïve T cells to form activated T cells characterized by the production of enhanced levels of Interferon gamma (IFNγ) and/or tumor necrosis factor α. (TNFα). The activated T cells can be administered to the animal in need of stimulation of a cytotoxic T cell response to the specific antigen.

Suitable antigens include, for example, a tumor specific antigen, a tumor associated antigen, a viral antigen, a bacterial antigen, tumor cells, bacterial cells, transformed or transfected cells expressing a recombinant antigen, wherein the antigen is a bacterial antigen, a viral antigen, a tumor associated or tumor specific antigen, a cell lysate, wherein the cell lysate is a bacterial cell lysate or a tumor cell lysate, a membrane preparation, wherein the membrane preparation is a bacterial cell or tumor cell membrane preparation, or a recombinantly produced antigen, a peptide antigen (e.g., a synthetic peptide antigen), or an isolated antigen, wherein the antigen is a bacterial, viral, tumor associated or tumor specific antigen. The immature dendritic cells can be simultaneously contacted with the predetermined antigen, the dendritic cell maturation agent with or without Interferon γ and the inflammation-activating lipid, or the immature dendritic cells can be contacted with the predetermined antigen prior to of subsequent contacting the cells with the dendritic cell maturation with or without Interferon γ and the inflammation-activating lipid.

In certain embodiments, the in vitro or ex vivo method can further include isolating monocytic dendritic cell precursors from the animal; and culturing the precursors in the presence of a dendritic cell differentiation agent to form the immature dendritic cells. The dendritic cell differentiation agent can be, for example, GM-CSF, or a combination of GM-CSF and IL-4, IL-13, or IL-15.

The immature dendritic cells and T cells can be autologous to the animal, or allogenic to the animal. Alternatively, the immature dendritic cells and T cells can have the same MHC haplotype as the animal or share an MHC marker. In certain embodiments, the animal can be human, or can be a non-human animal.

In another embodiment an in vitro or ex vivo method is provided for producing maturing isolated hyperactivated dendritic cells that can uptake and process antigen, and that can induce an enhanced anti-tumor response subsequent to administration to an individual. The method comprises providing non-activated immature dendritic cells; contacting the immature dendritic cells with an effective amount of dendritic cell maturation agent with or without Interferon γ, and an inflammation-activating lipid under in vitro or ex vivo culture conditions suitable for inducing maturation of the immature dendritic cells; and isolating the maturing dendritic cells prior to full maturation.

In certain embodiments of the above methods the inflammation-activating lipid used in the method can be one of more of oxidized 1-palmitoyl-2-arachidonyl-sn-glycero-3-phosphorylcholine (oxPAPC), 1-palmityl-2-arachidonyl-sn-glycero-3-phosphoryicholine (PAPC), 1-palmitoyl-2-glutaroyl-sn-glycero-3-phosphocholine (PGPC), and 1-palmitoyl-2-[5-oxovaleroyl]-sn-glycero-3-phosphorylcholine (POV-PC), and species of oxPAPC (e.g., 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC). The method can further comprise the steps of formulating the isolated maturing dendritic cells prior to full maturation followed by administration of the maturing dendritic cells to the individual. The method can also optionally include cryopreserving the maturing dendritic cells and thawing the maturing dendritic cells prior to formulation and administration.

In another embodiment an in vitro or ex vivo method is provided for producing maturing isolated hyperactivated dendritic cells that can uptake and process antigen, and that can induce an enhanced anti-tumor response subsequent to administration to an individual. The method comprises providing non-activated immature dendritic cells; contacting the immature dendritic cells in vitro or ex vivo with an effective amount of a dendritic cell maturation agent in combination with or without Interferon γ under culture conditions suitable for inducing maturation of the immature dendritic cells; and isolating the maturing dendritic cells prior to full maturation. The maturing dendritic cells are formulated for administration to an individual, for example, an individual with a solid tumor, together with an inflammation-activating lipid. The maturing dendritic cells can be combined with the inflammation-activating lipid in either the same formulation or it can be formulated separately. When formulated separately, the maturing dendritic cells and the inflammation-activating lipid can be administered either simultaneously or sequentially in either order. In certain embodiments, the inflammation-activating lipid can be formulated for topical administration and can be applied to the area of, for example, the skin or mucosa where the formulated maturing dendritic cells is to be administered.

In certain embodiments of the in vitro or ex vivo methods above the dendritic cell maturation agent is a Toll-like Receptor agonist, such as an agonist of TLR3, TLR4, TLR7 and/or TLR8, or TLR9 such as for example, a bacterial LPS, BCG, an imidazoquinoline compound, e.g., a imidazoquinoline-4-amine compound, such as 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (837, imiquimod), and their derivatives, a synthetic double stranded polyribonucleotide, e.g., poly I:C, or its derivative, poly [I]:poly[C(12)U], and the like, a sequence of nucleic acids containing unmethylated CpG motifs known to induce the maturation of DC, and the like, or any combination thereof.

The method can use as the inflammation-activating lipid an oxidized lipid, including for example, one or more of a bioactive oxidized phospholipid which can contain fragmentation products of polyunsaturated fatty acid (PUFA), such as 1-palmitoyl-2-oxovaleroyl-sn-glycero-3-phosphorylcholine and 9-keto-10-dodecendioic acid ester of 2-lyso-phosphatidyl choline (KOdiA-PC). Chromatographic separation of many products formed by oxidation of 1-palmitoyl-2-arachidonoyl-sn-glycero-3-phosphorylcholine (PAPC) led to the identification of 1-palmitoyl-2-(5-oxovaleroyl)-sn-glycero-3-phosphatidylcholine (POVPC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC) as potent lipid mediators of inflammation. In certain embodiments of the disclosure the oxidized lipid can include, but is not limited to oxPAPC which is a mixture of 1-palmitoyl-2-arachidonoyl-sn-glycero-3-phosphocholine (PAPC), 1-palmitoyl-2-glutaroyl-sn-glycero-3-phosphocholine (PGPC), and 1-palmitoyl-2-[5-oxovaleroyl]-sn-glycero-3-phosphorylcholine (POVPC), and species of oxPAPC (e.g., 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC), as well as Rhodo LPS (LPS-RS or LPS from Rhodobacter sphaeroides, which has been previously demonstrated to activate a caspase 11-dependent inflammasome).

Predetermined antigen useful in the present method include a tumor specific antigen, a tumor associated antigen, a viral antigen, a bacterial antigen, a tumor cell, a bacterial cell, a transformed or transfected cell expressing a recombinant protein, wherein the recombinant protein is a bacterial, viral, tumor associated or tumor specific protein, a bacterial or tumor cell lysate, a bacterial or tumor cell membrane preparation, and a recombinantly produced antigen, a peptide antigen, or an isolated antigen, wherein the antigen, peptide antigen or isolated antigen can be a bacterial, viral, tumor associated, or tumor specific antigen. In certain embodiments, the recombinantly produced antigen, peptide antigen or isolated antigen is a bacterial, a viral, a tumor associated, or a tumor specific antigen, peptide antigen, or isolated antigen. In these embodiments none of the predetermined antigens are intended to be dendritic cell maturation agents.

In an optional embodiment, the method can further comprise isolating monocytic dendritic cell precursors; and culturing the precursors in vitro or ex vivo in the presence of a dendritic cell differentiating agent to form the immature dendritic cells. The dendritic cell differentiating agent can be GM-CSF when used alone with a high concentration of an animal protein, for example, a bovine, goat, horse, monkey, ape, and/or human protein, particularly when used in combination with a culture media supplemented with a high protein concentration, or a combination of GM-CSF and Interleukin 4 (IL-4), Interleukin 7 (IL-7), Interleukin 13 (IL-13), or Interleukin 15 (IL-15). The monocytic dendritic cell precursors can be isolated from a human subject.

In yet another embodiment the present disclosure provides an in vitro method for producing an enhanced Th1 immune response to an antigen. The method comprises: providing immature dendritic cells; contacting the immature dendritic cells in vitro or ex vivo with an effective amount of a dendritic cell maturation agent, such as a TLR agonist, with or without Interferon gamma (IFNγ), and a predetermined antigen under culture conditions suitable for maturation of the immature dendritic cells to form the mature dendritic cell population; wherein the maturing dendritic cells take up and process antigen during maturation; formulating the mature dendritic cells for administration to an individual; and administering the mature dendritic cell formulation together with an effective amount of an inflammation-activating lipid to produce an enhanced Th1 immune response to the predetermined antigen as compared with the formulated dendritic cell composition administered without the inflammation-activating lipid.

In this embodiment, the dendritic cell maturation agent can be one of, or a combination of Toll-like receptor (TLR) agonists, such as, agonists of TLR-3, TLR-4, TLR-7 and/or TLR-8, and TLR9, for example and not for limitation, a bacterial LPS, BCG, an imidazoquinoline compound, e.g., a imidazoquinoline-4-amine compound, such as 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837, Imiquimod), and their derivatives, a synthetic double stranded polyribonucleotide, e.g., poly I:C; or its derivative poly [I]:poly[C(12)U], a sequence of nucleic acids containing unmethylated CpG motifs known to induce the maturation of DC, for example, ODN 2216 (5′-ggGGGACGA:TCGTCgggggg-3′), and ODN2336 (5′-gggGGACGAC:GTCG TGgggggg-3′), and the like, or any combination thereof.

Inflammation-activating lipids useful in this method can be a bioactive phospholipid which can contain fragmentation products of, one or more of a polyunsaturated fatty acid (PUFA), such as 1-palmitoyl-2-oxovaleroyl-sn-glycero-3-phosphorylcholine and 9-keto-10-dodecendioic acid ester of 2-lyso-phosphatidyl choline (KOdiA-PC). Chromatographic separation of many products formed by oxidation of 1-palmitoyl-2-arachidonoyl-sn-glycero-3-phosphorylcholine (PAPC) led to the identification of 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC) as potent lipid mediators of inflammation. In certain embodiments of the disclosure the oxidized lipid can include, but is not limited to oxPAPC which is a mixture of 1-palmitoyl-2-arachidonoyl-sn-glycero-3-phosphocholine (PAPC), 1-palmitoyl-2-glutaroyl-sn-glycero-3-phosphocholine (PGPC), and 1-palmitoyl-2-[5-oxovaleroyl]-sn-glycero-3-phosphorylcholine (POV-PC), and species of oxPAPC (e.g., 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

In addition, the predetermined antigen can be a tumor specific antigen, a tumor associated antigen, a viral antigen, a bacterial antigen, a tumor cell, a bacterial cell, a transformed or transfected cell producing a recombinant protein, wherein the recombinant protein is a bacterial protein, a viral protein, a tumor specific, or tumor associate protein, a bacterial or tumor cell lysate, a bacterial or tumor cell membrane preparation, a recombinantly produced antigen, a peptide antigen, or an isolated antigen. The recombinantly produced antigen, peptide antigen or isolated antigen can be a bacterial, a tumor associated, or a tumor specific antigen, peptide antigen, or isolated antigen.

The in vitro or ex vivo method for producing an enhanced immune response above, can further comprise isolating monocytic dendritic cell precursors and culturing the precursors in vitro or ex vivo in the presence of a dendritic cell differentiating agent to form the immature dendritic cells. Dendritic cell differentiating agents useful in the method include, for example, GM-CSF, or a combination of GM-CSF and Interleukin 4 (IL-4), Interleukin 7 (IL-7), Interleukin 13 (IL-13), or Interleukin 15 (IL-15). The monocytic dendritic cell precursors can be isolated from a human subject.

In certain embodiments, the formulated mature dendritic cells and the inflammation-activating lipid are administered simultaneously or sequentially in any order.

In another embodiment the in vitro or ex vivo method comprises maturation of immature dendritic cells (DC) and for activating those cells without the use of a dendritic cell maturation agent. The activated DC can be used for inducing an antigen specific T cell response. Methods can also comprise the addition of a directional maturation agent, such as interferon gamma (IFNγ), to induce a Th-1 and/or Th-2 bias in the response obtained. Unlike prior methods, activation of the immature dendritic cells in the method of the present disclosure is initiated or triggered by contacting isolated immature dendritic cells with a new, clean and unused tissue culture substrate and a dendritic cell differentiation inducing agent under conditions suitable for adherence of the dendritic cell to the tissue culture surface. In a typical method of the present disclosure, activation is achieved without the addition of a maturation agent. The immature dendritic cells are removed from a prior culture substrate or purification media and isolated from the prior culture media. The isolated immature dendritic cells are then counted and frozen for later use or combined with a fresh culture media without adding a dendritic cell maturation factor for the remainder of the process.

In one alternative method, a directional maturation agent can be added during activation to bias the dendritic cells so that they can polarize a T cell response to a Th-1 or a Th-2 response. As an example, but not as a limitation to the disclosed methods, the agent used is IFNγ which can be added to bias the T cell response toward a Th-1 response. Interferon gamma (IFNγ) added in vitro or ex vivo to monocytic dendritic cell precursors or immature DCs by itself does not induce DC differentiation and/or maturation.

Tissue culture substrates useful in the methods of this embodiment can comprise a tissue culture well, flask, bottle, bag, or any matrix used in a bioreactor, such as fiber, beads, plates, and the like. Typically, tissue culture substrates comprise plastics, such as polystyrene, Teflon® (polytetrafluoroethylene, PTFE), and the like. These substrates are uncoated with proteins or other substituents to increase the binding of various cells to the substrate. Those tissue culture substrates most often used for the ex vivo culture of dendritic cells for immunotherapy comprise tissue culture-flasks, -bags, or cell fractions which are comprised of multiple stacked layers of plastic, and the like.

Fully mature dendritic cells differ qualitatively and quantitatively from immature DCs. Fully mature DCs express higher levels of MHC class I and class II antigens, and of T cell costimulatory molecules, i.e., CD80 and CD86. These changes typically increase the capacity of the dendritic cells to activate T cells, for example, by increasing antigen density on the cell surface, as well as the T cell activation signal through the counterparts of the costimulatory molecules on the T cells, e.g., like CD28. In addition, mature DC produce large amounts of cytokines, which stimulate and direct the T cell response. Two of these cytokines are Interleukin 10 (IL-10) and Interleukin (IL-12). These cytokines have opposing effects on the direction of the induced T cell response. IL-10 production results in the induction of a Th-2 type response, while IL-12 production results in a Th-1 type response.

In one embodiment, the hyperactive mature dendritic cells produced by any of the above methods are used for producing a medicament for treating cancer, particularly a solid tumor, a bacterial infection, or a viral infection. The medicament can be a formulation of the hyperactive mature dendritic cells comprising a therapeutically effective amount of the cells. The formulation can comprise greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or even greater than 10¹¹. Certain embodiment of the compositions and formulations thereof can be adapted for administration by, for example, injection, infusion, perfusion, or lavage and can more particularly include administration through one or more means such as, for example, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous infusions and/or bolus injections.

These and other aspects of the present disclosure will become more readily apparent to those possessing ordinary skill in the art when reference is made to the following detailed description

### DETAILED DESCRIPTION

The present disclosure provides in vitro or ex vivo methods for inducing maturation of immature dendritic cells (DC) and for priming those cells for an enhanced antigen-specific cytotoxic T cell response (Th-1 response). The present disclosure also provides dendritic cell populations useful for activating and for preparing T cell populations polarized towards enhanced production of type 1 cytokines (e.g., IFNγ, TNFα, IL-1β, and/or IL2). Such dendritic cell populations include immature monocytic dendritic cells contacted in vitro or ex vivo with a Toll-like Recentor (TLR) agonist, such as, for example, a bacterial lipopolysaccharide, such as E. coli LPS, BCG, Poly I:C and its less toxic derivatives, and/or R848 or R837, with or without Interferon γ, and an inflammation-activating lipid. Optionally the DCs can also be contacted with a predetermined antigen under suitable maturation conditions. The immature dendritic cells can be contacted with the antigen either simultaneous with, prior or subsequent to maturation. Where contact with the predetermined antigen takes place after maturation or where the ability to take up antigen has been substantially reduced, antigen uptake can be enhanced using known methods, such as osmotic loading.

Alternatively, immature monocytic dendritic cells, already exposed to antigen (e.g., in vivo), can be contacted with the TLR agonist with or without Interferon γ, and the inflammation-activating lipid under suitable maturation conditions. The resulting hyperactivated mature dendritic cells are primed to activate an immune response and to activate and potentially polarize T cells (e.g., naïve T cells) towards a type 1 response. A type 1 response includes production of type 1 cytokines (e.g., IFNγ, and/or IL-2), production of more IL-12 p70, a cytotoxic T cell response, production of Th-1 cells, an increase of effector and memory T cells, the production of an increase in terminal differentiation and/or activation of CD4⁺ and CD8⁺ T cells, and the production of certain types of antibodies. The upregulation of IL-1β and tumor Necrosis Factor α (TNFα) can also be enhanced. In contrast, a type 2 response is characterized by production of IL-4, IL-5 and IL-10, production of more IL-10 than IL-12 p70, production of Th-2 cells, and lack of induction of a CTL response.

In a related aspect, compositions are provided comprising immature dendritic cells, such as enriched cell populations of CD34⁺ hematopoietic stem cells, or monocytic dendritic cell precursors, which can also prime those dendritic cells for an enhanced type 1 response. Such mature, primed monocytic dendritic cells can increase Major Histocompatibility Complex (MHC) class-I presentation of a predetermined antigen, i.e., a predetermined exogenous antigen. MHC class-I (MHC-I) presentation of antigen is desired to induce differentiation of cytotoxic T lymphocytes (CTL) and stimulation of antigen-specific CTL-mediated lysis of target cells. Such compositions include TLR agonists, such as, for example, LPS, BCG, Poly I:C, and R848, with or without IFNγ which can be admixed with a cell population comprising immature dendritic cells, to mature the immature dendritic cells, and in the case of BCG, to convert or overcome the inhibition of IL-10 induced by contact of the immature dendritic cells with BCG. Additionally, such compositions can comprise an inflammation-activating lipid. Immature dendritic cells contacted with such compositions undergo enhanced maturation and activation and typically produce greater amounts of biologically active IL-12, TNFα, and/or IL-1, (e.g., IL-10), as compared with an immature dendritic cell population contacted with the TLR agonist, such as LPS, BCG, Poly I:C, and/or R848, with or without IFNγ, and without the inflammation-activating lipid.

In another aspect, monocytic dendritic cell precursors obtained from subjects or donors can be contacted with cytokines (e.g., GM-CSF alone with a high concentration of, for example, an animal protein (human serum albumin), or GM-CSF in combination with, for example, IL-4, IL-7, IL-13 or IL-15) to obtain immature dendritic cells. The immature dendritic cells can then be contacted with a predetermined antigen, either in combination with the TLR agonist; optionally with or without Interferon γ; and the inflammation-activating lipid alone, or in combination with an additional cytokine, to mature the dendritic cells and to prime the cells for inducing an enhanced type 1 immune response in T cells. In certain embodiments, MHC Class-I antigen processing is stimulated, which is useful to elicit a CTL response against cells displaying the predetermined antigen.

Dendritic cells are a diverse population of antigen presenting cells found in a variety of lymphoid and non-lymphoid tissues. (See Liu, Cell 106:259-62 (2001); Steinman, Ann. Rev. Immunol. 9:271-96 (1991)). Dendritic cells include lymphoid dendritic cells of the spleen, Langerhans cells of the epidermis, and veiled cells in the blood circulation. Collectively, dendritic cells are classified as a group based on their morphology, high levels of surface MHC-class II expression, and absence of certain other surface markers expressed on T cells, B cells, monocytes, and natural killer cells. In particular, monocyte-derived dendritic cells (also referred to as monocytic dendritic cells) usually express CD11a, CD80, CD86, and are HLA-DR⁺, but are CD14⁻.

In contrast, monocytic dendritic cell precursors (typically monocytes) are usually CD14⁺. Monocytic dendritic cell precursors can be obtained from any tissue where they reside, particularly lymphoid tissues such as the spleen, bone marrow, lymph nodes, and thymus. Monocytic dendritic cell precursors also can be isolated from the circulatory system. Peripheral blood is a readily accessible source of monocytic dendritic cell precursors. Umbilical cord blood is another source of monocytic dendritic cell precursors. Monocytic dendritic cell precursors can be enriched from or isolated from a variety of organisms in which an immune response can be elicited. Such organisms include animals, for example, including humans, and non-human animals, such as, primates, mammals (including dogs, cats, mice, and rats), birds (including chickens), as well as transgenic species thereof.

In certain embodiments, the monocytic dendritic cell precursors and/or immature dendritic cells can be enriched or isolated from a healthy subject or from a subject in need of immunostimulation, such as, for example, a cancer patient or other subject for whom cellular immunostimulation can be beneficial or desired (i.e., a subject having a bacterial or viral infection, and the like). Dendritic cell precursors and/or immature dendritic cells also can be obtained as an isolated or enriched cell population from an HLA-matched healthy individual for administration to an HLA-matched subject in need of immunostimulation.

Dendritic Cell Precursors and Immature Dendritic Cells

Cell populations enriched for dendritic cell precursors, immature dendritic cells, or even mature dendritic cells can comprise significantly enriched cell populations comprising greater than 95 to 98% or more of the desired cells. While in other embodiments, the enriched cell population only increases the desired cells by 5 to 10% above the number of cells in the original cell population. It is preferred that the enriched cell population comprises as many of the desired cells as possible with the methods used.

Methods for obtaining cell populations enriched for dendritic cell precursors and immature dendritic cells from various sources, including blood and bone marrow, are known in the art. For example, enriched cell populations of dendritic cell precursors and immature dendritic cells can be isolated by collecting heparinized blood, by apheresis or leukapheresis, by preparation of buffy coats, rosetting, centrifugation, density gradient centrifugation (e.g., using Ficoll (such as FICOLL-PAQUE®), PERCOLL® (colloidal silica particles (15-30 mm diameter) coated with non-dialyzable polyvinylpyrrolidone (PVP)), sucrose, and the like), differential lysis of cells, filtration, and the like. In certain embodiments, a leukocyte population can be prepared, such as, for example, by collecting blood from a subject, defibrinating to remove the platelets and lysing the red blood cells. Dendritic cell precursors and immature dendritic cells can optionally be enriched for monocytic dendritic cell precursors by, for example, centrifugation through a PERCOLL® gradient.

Enriched cell populations of dendritic cell precursors and immature dendritic cells optionally can be prepared in a closed, aseptic system. As used herein, the terms “closed, aseptic system” or “closed system” refer to a system in which exposure to non-sterilize, ambient, or circulating air or other non-sterile conditions is minimized or eliminated. Closed systems for isolating cell populations enriched for dendritic cell precursors and immature dendritic cells generally exclude density gradient centrifugation in open top tubes, open air transfer of cells, culture of cells in tissue culture plates or unsealed flasks, and the like. In a typical embodiment, the closed system allows aseptic transfer of the dendritic cell precursors and immature dendritic cells from an initial collection vessel to a sealable tissue culture vessel without exposure to non-sterile air.

In certain embodiments, cell populations enriched for monocytic dendritic cell precursors are isolated by adherence to a monocyte-binding substrate, as disclosed in WO 2003/010292, the disclosure of which is incorporated by reference herein. For example, a population of leukocytes (e.g., isolated by leukapheresis) can be contacted with a monocytic dendritic cell precursor adhering substrate. When the population of leukocytes is contacted with the substrate, the monocytic dendritic cell precursors in the leukocyte population preferentially adhere to the substrate. Other leukocytes (including other potential dendritic cell precursors) exhibit reduced binding affinity to the substrate, thereby allowing the monocytic dendritic cell precursors to be preferentially enriched on the surface of the substrate.

Suitable substrates include, for example, those having a large surface area to volume ratio. Such substrates can be, for example, a particulate or fibrous substrate. Suitable particulate substrates include, for example, glass particles, plastic particles, glass coated plastic particles, glass-coated polystyrene particles, and other beads suitable for protein absorption. Suitable fibrous substrates include microcapillary tubes and microvillous membrane. The particulate or fibrous substrate usually allows the adhered monocytic dendritic cell precursors to be eluted without substantially reducing the viability of the adhered cells. A particulate or fibrous substrate can be substantially non-porous to facilitate elution of monocytic dendritic cell precursors or dendritic cells from the substrate. A “substantially non-porous” substrate comprises a substrate in which at least a majority of pores present in the substrate are smaller than the cells to minimize entrapping cells in the substrate.

Adherence of the monocytic dendritic cell precursors to the substrate can optionally be enhanced by addition of binding media. Suitable binding media include monocytic dendritic cell precursor culture media (e.g., AIM-V®, RPMI 1640, DMEM, X-VIVO 15®, and the like) supplemented, individually or in any combination, with for example, cytokines (e.g., Granulocyte/Macrophage Colony Stimulating Factor (GM-CSF), or Interleukin 13 (IL-13)), blood plasma, serum (e.g., human serum, such as autologous or allogenic sera), purified proteins, such as serum albumin, divalent cations (e.g., calcium and/or magnesium ions) and other molecules that aid in the specific adherence of monocytic dendritic cell precursors to the substrate, or that prevent adherence of non-monocytic dendritic cell precursors to the substrate. In certain embodiments, the blood plasma or serum can be heated-inactivated. The heat-inactivated plasma can be autologous or heterologous to the leukocytes.

Following adherence of monocytic dendritic cell precursors to the substrate, the non-adhering leukocytes are separated from the monocytic dendritic cell precursor/substrate complexes. Any suitable means can be used to separate the non-adhering cells from the complexes. For example, the mixture of the non-adhering leukocytes and the complexes can be allowed to settle, and the non-adhering leukocytes and media decanted or drained. Alternatively, the mixture can be centrifuged, and the supernatant containing the non-adhering leukocytes decanted or drained from the pelleted complexes.

In another embodiment the enriched population of monocytic dendritic cell precursors can be obtained by tangential flow filtration. See WO 2004/000444, incorporated herein by reference in its entirety. Using this method, the monocytic dendritic cell precursors are not activated and certain cytokines, such as IL-4 are not required to prevent the monocytic dendritic cell precursors from differentiating into macrophage during culture in vitro or ex vivo.

Cell populations enriched for dendritic cell precursors can be cultured in vitro or ex vivo for differentiation, maturation and/or expansion. (As used herein, isolated immature dendritic cells, dendritic cell precursors, T cells, and other cells, refers to cells that, by human hand, exists apart from their native environment, and are therefore not a product of nature. Isolated cells can exist in purified form, in semi-purified form, such as enriched cell populations, or in a non-native environment.)

Briefly, in vitro or ex vivo differentiation typically involves culturing dendritic cell precursors, or populations of cells having dendritic cell precursors, in the presence of one or more differentiation agents. Suitable differentiating agents can be, for example, cellular growth factors (e.g., cytokines such as (GM-CSF), and combinations of GM-CSF and Interleukin 4 (IL-4), Interleukin 7 (IL-7), Interleukin 13 (IL-13), or Interleukin 15 (IL-15). In certain embodiments, the monocytic dendritic cells precursors are differentiated to form monocyte-derived immature dendritic cells.

The dendritic cell precursors can be cultured and differentiated in suitable in vitro or ex vivo culture conditions. Suitable tissue culture media include AIM-V®, RPMI 1640, DMEM, X-VIVO 15®, and the like. The tissue culture media can be supplemented with serum, amino acids, vitamins, cytokines, such as GM-CSF or GM-CSF in combination with IL-4, divalent cations, and the like, to promote differentiation of the dendritic cell precursors. In certain embodiments, the dendritic cell precursors can be cultured in a serum-free media. Such culture conditions can optionally exclude any animal-derived products. A typical cytokine combination in a typical dendritic cell culture medium is about 500 units/ml each of GM-CSF and IL-4.

Dendritic cell precursors, when differentiated to form immature dendritic cells are phenotypically similar to skin Langerhans cells. Immature dendritic cells typically are CD14⁻ and CD11c⁺, express low levels of CD86 and CD83, and can capture or uptake soluble antigens via specialized endocytosis.

The immature dendritic cells can be matured to form mature hyperactive dendritic cells. Mature DCs lose the ability to take up antigen and display up-regulated expression of costimulatory cell surface molecules and various cytokines. Specifically, mature DCs express higher levels of MHC class I and II antigens than immature dendritic cells, and mature dendritic cells are generally identified as being CD80⁺, CD83⁺, CD86⁺ and CD14⁻. Greater MHC expression leads to an increase in antigen density on the DC surface, while up regulation of costimulatory molecules CD80 and CD86 strengthens the T cell activation signal through the counterparts of the costimulatory molecules, such as CD28 on the T cells.

Mature hyperactive dendritic cells of the present invention can be prepared (i.e., matured) by contacting the immature dendritic cells with effective amounts or concentrations of a TLR agonist, such as, for example, a bacterial lipopolysaccharide, such as LPS; whole Bacillus Calmette-Guerin (BCG or derivatives thereof, a double stranded RNA, such as Poly I:C or its less toxic derivatives, such as Poly I:C(12)U, or R848, with or without Interferon γ followed by activation with an inflammation-activating lipid. Effective amounts of the TLR agonist, such as for example the bacteria BCG, typically range from about 10⁵ to 10⁷ cfu per milliliter of tissue culture media. Effective amounts of IFNγ typically range from about 100-1000 U per milliliter of tissue culture media. Bacillus Calmette-Guerin (BCG) is an avirulent strain of Mycobacterium bovis. As used herein, BCG refers to whole BCG as well as cell wall constituents, BCG-derived lipoarabidomannans, and other BCG components that are associated with induction of a type 2 immune response. The bacteria, or bacterial component, such as BCG, is optionally inactivated, such as heat-inactivated BCG, formalin-treated BCG, and the like.

Maturing the immature dendritic cells with the TLR agonist with or without Interferon γ and activation with the inflammation-activating lipid promotes the formation of hyperactive DCs which can produce enhanced levels of IL-12 and/or IL-1, more specifically IL-1β, and reduces or inhibits production of IL-10, thereby priming the mature dendritic cells for inducing a type 1(Th-1) response.

The immature DC are typically contacted with effective amounts of the TLR agonist with or without Interferon γ for about one hour to about 24 hours. Subsequently the inflammation-activating lipid can be added. In a typical embodiment, the inflammatory-activating lipid is added about 3 hours after the induction of maturation, although this time can be extended to 4 hours, 5 hours, 6 hours, 7 hours, 8 hours, 10 hours, and up to 24 or more hours. The immature dendritic cells can be cultured and matured in suitable maturation culture conditions. Suitable tissue culture media include AIM-V®, RPMI 1640, DMEM, X-VIVO 15®, and the like. The tissue culture media can be supplemented with amino acids, vitamins, cytokines, such as GM-CSF and/or IL4, IL13, or IL15, divalent cations, and the like, to promote maturation of the cells. A typical cytokine combination is about 500 units/ml each of GM-CSF and IL4.

In another in vitro or ex vivo method for inducing the formation of immature dendritic cells, monocytic dendritic cell precursors in a cell population enriched for the precursors are differentiated to form immature dendritic cells in the presence of GM-CSF and IL-4. In this embodiment, the immature dendritic cells are collected from the tissue culture system, washed, counted and combined in vitro or ex vivo with a predetermined antigen in a new, clean, uncoated tissue culture vessel. The immature dendritic cells can be cultured in the presence of a predetermined soluble or particulate antigen under conditions typical for dendritic cell maintenance in a typical dendritic cell culture media supplemented with GM-CSF and IL-4. No dendritic cell maturation agent is added to the media and it should be noted that any added predetermined antigen is not considered a dendritic cell maturation agent. Maturation of the cells can be determined using well known methods in the art including determining the presence of cell surface markers characteristic of mature dendritic cells.

Cell surface markers can be detected in assays familiar to the art, such as flow cytometry, immune histochemistry, and the like. The cells can also be monitored for cytokine production (e.g., by ELISA, FACS, or another immune assay). In a DC population matured according to the present invention, IL12 levels are higher than the IL12 levels produced by mature dendritic cells induced to mature from immature dendritic cell by the TLR agonists of the present disclosure with or without IFNγ without the inflammation-activating lipid, to promote an enhanced type 1 (Th-1) response. For example, the mature hyperactive DCs can produce an enhanced amount of biologically active IL12, IL-2, TNFα and/or IL1 (IL-1β); show an increase in terminal differentiation and/or activation of CD4⁺ and CD8⁺ T cells, and an increase in effector and memory T cells. Mature DCs also lose the ability to uptake antigen by pinocytosis, which can be analyzed by uptake assays familiar to one of ordinary skill in the art.

Dendritic cell precursors, immature dendritic cells, and mature dendritic cells, either primed or unprimed, with antigens can be cryopreserved for use at a later date. Methods for cryopreservation are well-known in the art. See, for example, U.S. Pat. No. 5,788,963 incorporated herein by reference in its entirety.

TLR Agonists

The activators of the present disclosure include those the stimulate Pattern Recognition Receptor (PRR)-dependent cellular responses. Pattern Recognition Receptors include Toll-like Receptors (TLRs), RIG-1 like Receptors (RLRs), NOD-like receptors (NLR) and C-type Lectin Receptors (CLRS). PRRs act to either directly or indirectly detect molecules that are common to broad classes of microbes. These molecules are classically referred to as pathogen associated molecular patterns and include factors such as bacterial lipopolysaccharide (LPS), bacterial flagellin, or viral double stranded RNA. TLRs, as above, are a class of pattern recognition receptors (PRRs), which detect microbial molecules classically referred to as pathogen associated molecular patterns (PAMPs). The TLR immune receptors are expressed on the membranes of leukocytes, including dendritic cells, and the binding of TLR agonists triggers molecular events that can lead to an immune response and antigen-specific acquired immunity. Many TLR agonists are also dendritic cell maturation agents as discussed below. Bacterial lipopolysaccharide (LPS) is a strong agonist of TLR4 with the ability to enhance immune responses to soluble antigens. LPS stimulates immune system cells by the TLR4 pathway, which recognizes common PAMPs. Additional TLR agonists include synthetic molecules, such as for example, Poly I:C which binds TLR3, Pam2CSK4 (Pam2) which binds and activates the TLR 2 and TLR 6 pathways, Pam3CSK4 (Pam3) which binds and activates the TLR 2 and TLR1 pathways, and R848 and R837 which are imidazoquinolines which bind and activate the TLR 7 and TLR 8 pathways. Unmethylated CpG motif sequences (oligodeoxynucleotides (ODNs) common to certain bacteria bind to and activate TLR9.

In specific embodiments of the methods described herein the TLR agonist can be a bacterial or microbial product and be a bacterial LPS, such as, for example, an E. coli LPS or an LPS derived or isolated from any other bacterial that have an LPS (e.g., monophosphoryl lipid A (MPLA)), bacterial cell membranes or cell wall skeleton, and the like. The bacteria or bacterial product can also be, for example, another TLR4 agonist BCG or products of BCG including, for example, cell wall constituents, BCG-derived lipoarabidomannans, and other BCG products. BCG is optionally inactivated, such as heat-inactivated BCG, formalin-treated BCG, or by a combination of heat and other inactivation methods. In other examples the TLR agonist can be Poly I:C, Pam2CSK4, Pam3CSK4, R848, R837, or an ODN where the optimal CpG motif for human appears to be GTCGTT.

Dendritic Cell Maturation Agents

Dendritic cell maturation agents can include, for example, but are not limited to, TLR agonists that bind to preferably TLR2, TLR4, TLR3, TLR7 and/or TLR8. BCG is a TLR2 and TLR4 agonist; LPS is a TLR4 agonist; imidazoquinoline compounds are TLR7 and TLR8 agonists; a synthetic double stranded polyribonucleotide, e.g., Poly I:C and its derivative poly[I]:poly[C(12)U], are TLR3 agonists. Imidazoquinoline compounds that can act as dendritic cell maturation agents include, imidazoquinoline-4-amine compounds, such as 4-amino-2-ethoxymethyl-α,α-dimethyl-1H-imidazol[4,5-c]quinolin-1-ethanol (designated R848) or 1-(2-methylpropyl)-1H-imidazo[4,5-c]quinolin-4-amine (designated as R837), and their derivatives (See, for example, WO2000/47719, incorporated herein by reference in its entirety), Other agonists of a Toll-like receptor (TLR) that can induce the maturation of immature dendritic cells include for example, agonists of TLR9, such as, for example, a sequence of nucleic acids containing unmethylated CpG motifs known to induce the maturation of DC. Examples of such CpG motifs that can induce DC maturation include, for example, ODN 2216 (5′-ggGGGACGA:TCGTCgggggg-3′), and ODN2336 (5′-gggGGACGAC:GTCG TGgggggg-3′), and the like, or any combination thereof.

As a specific example, effective amounts of BCG typically range from an equivalent to about 10⁵ to 10⁷ cfu per milliliter of tissue culture media prior to deactivation. As above, the BCG can be whole BCG as well as cell wall constituents, BCG-derived lipoarabidomannans, and other BCG components. BCG is optionally inactivated, such as heat-inactivated BCG, formalin-treated BCG, or by combinations of heat and other inactivation methods, and the like.

As above, the immature dendritic cells can be cultured with the dendritic cell maturation agent for a time period sufficient for the dendritic cells to mature. Once the dendritic cells are fully mature, they lose to ability to efficiently uptake and process antigen for presentation. In addition, fully mature dendritic cells have an increased expression of co-stimulatory molecules CD80, CD86, CD83, MHC-I and MHC-II, and the like.

Inflammation-Activating Lipids

The term “inflammation-activating lipid,” as used herein, refers to a lipid that can elicit an inflammatory response in a caspase 11 (Caspase 4/5 in humans)-dependent inflammasome of a cell. Exemplary inflammation-activating lipids include oxidized phospholipids. Oxidized phospholipids are generated by the oxidation of polyunsaturated fatty acid residues, which are usually present in the phospholipids at the sn-2 position. Oxidation of phospholipids can be initiated either enzymatically or by reactive oxygen species and propagates via the classical mechanism of lipid peroxidation chain reaction. Bioactive oxidized phospholipids can contain fragmentation products of a polyunsaturated fatty acid (PUFA), such as 1-palmitoyl-2-oxovaleroyl-sn-glycero-3-phosphorylcholine and 9-keto-10-dodecendioic acid ester of 2-lyso-phosphatidyl choline (KOdiA-PC). Chromatographic separation of many products formed by oxidation of 1-palmitoyl-2-arachidonoyl-sn-glycero-3-phosphorylcholine (PAPC) led to the identification of 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC) as potent lipid mediators of inflammation. In certain embodiments of the disclosure the oxidized lipid can include, but is not limited to oxPAPC which is a mixture of 1-palmitoyl-2-arachidonoyl-sn-glycero-3-phosphocholine (PAPC), 1-palmitoyl-2-glutaroyl-sn-glycero-3-phosphocholine (PGPC), and 1-palmitoyl-2-[5-oxovaleroyl]-sn-glycero-3-phosphocholine (POVPC), and species of oxPAPC (e.g., 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC), as well as Rhodo LPS (LPS-RS or LPS from Rhodobacter sphaeroides, which has been previously demonstrated to activate a caspase 11-dependent inflammasome).

The inflammation-activating lipid can be added to the maturing dendritic cells about 3 hours after the induction of maturation, although this time can be extended to about 4 hours, about 5 hours, about 6 hours, about 7 hours, about 8 hours, about 10 hours, and up to about 24 or more hours. Incubation with the inflammation-activating lipid can be at least about 5 to about 18 hours and up to about 48 hours, or more. Typically, the maturing dendritic cells are cultured with the inflammation-activating lipid at least about 5 hours and can be for about 18 hours to about 24 hours, or more. The lipids can be used at concentrations of at least 1 μg/ml and up to 90 μg/ml or more. In certain embodiments the inflammation-activating lipid is used at about 1 μg/ml, about 3 μg/ml, about 10 μg/ml, about 25 μg/ml, about 30 μg/ml, about 90 μg/ml, about 100 μg/ml, or more.

Antigens

The mature, hyperactivated dendritic cells according to the present disclosure can present antigen to T cells. Mature, hyperactivated dendritic cells can be formed by contacting in vitro or ex vivo immature dendritic cells with a predetermined antigen either prior to, during or subsequent maturation.

Alternatively, immature dendritic cells that have already been contacted with antigen (e.g., in vivo prior to isolation) can be contacted in vitro or ex vivo with a composition comprising the TLR agonist with or without Interferon γ and the inflammation-activating lipid to form mature dendritic cells primed and hyperactivated for inducing an enhanced type 1 (Th-1) response.

Suitable predetermined antigens can include any antigen for which T-cell activation is desired. Such antigens can include, for example, bacterial antigens, tumor specific or tumor associated antigens (e.g., whole bacterial or tumor cells, tumor cell lysate, isolated antigens from tumors, fusion proteins, liposomes, and the like), viral antigens, and any other antigen or fragment of an antigen, e.g., a peptide or polypeptide antigen comprising a tumor associated or tumor specific epitope. In certain embodiments, the antigen can be, for example, but not limited to, a tumor cell lysate, a glioma tumor antigen, prostate specific membrane antigen (PSMA), prostatic acid phosphatase (PAP), or prostate specific antigen (PSA). (See, e.g., Pepsidero et al., Cancer Res. 40:2428-32 (1980); McCormack et al., Urology 45:729-44 (1995).) The antigen can also be a bacterial cell, bacterial lysate, tumor or bacterial cell or membrane fragment from a cellular lysate, or any other source known in the art. The antigen, such as a tumor, bacterial, or viral antigen, can be expressed or produced recombinantly, or even chemically synthesized. The recombinant antigen can also be expressed on the surface of a host cell (e.g., bacteria, yeast, insect, vertebrate or mammalian cells), can be present in a lysate, or can be purified from the lysate.

Antigen can also be present in a sample from a subject. For example, a tissue sample from a hyperproliferative or other condition in a subject can be used as a source of antigen. Such a sample can be obtained, for example, by biopsy or by surgical resection. Such an antigen can be used as whole cell, cell lysate, cell membrane preparation, or as an isolated antigen preparation.

Alternatively, a membrane preparation of cells of a subject (e.g., a cancer patient), or an established cell line, such as a tumor cell line, also can be used as an antigen or source of antigen.

In an exemplary embodiment, a glioma, prostate, breast, colon, pancreas, lung, or other tumor, or other tumor cells recovered from a surgical specimen can be used as a source of antigen. For example, a sample of a cancer patient's own tumor, obtained at biopsy or at surgical resection, can be used directly to present antigen to dendritic cells or to provide a cell lysate for antigen presentation. Alternatively, a membrane preparation of tumor cells of a cancer patient can be used. The tumor cell can be prostatic, lung, ovarian, colon, brain, melanoma, or any other type of tumor cell. Lysates and membrane preparations can be prepared from isolated tumor cells by methods known in the art.

In another exemplary embodiment, purified or semi-purified prostate specific membrane antigen (PSMA, also known as PSM antigen), which specifically reacts with monoclonal antibody 7E1 1-C.5, can be used as antigen. (See generally Horoszewicz et al., Prag. Clin. Biol. Res. 37:115-32 (1983), U.S. Pat. Nos. 5,162,504; 5,788,963; Feng et al., Proc. Am. Assoc. Cancer Res. 32: (Abs. 1418)238 (1991); the disclosures of which are incorporated by reference herein.) In yet another exemplary embodiment, an antigenic peptide having the amino acid residue sequence Leu His Glu Thr Asp Ser Ala Val (SEQ ID NO:1) (designated PSM-P1), which corresponds to amino acid residues 4-12 of PSMA, can be used as an antigen. Alternatively, an antigenic peptide having the amino acid residue sequence Ala Leu Phe Asp Ile Glu Ser Lys Val (SEQ ID NO:2) (designated PSM-P2), which corresponds to amino acid residues 711-719 of PSMA, can be used as antigen.

In a particular embodiment, an antigenic peptide having an amino acid residue sequence Xaa Leu (or Met) Xaa Val (or Leu) (designated PSM-PX), where Xaa represents any amino acid residue, can be used as antigen. This peptide resembles the HLA-A0201 binding motif, i.e., a binding motif of 9-10 amino acid residues with “anchor residues”, leucine and valine found in HLA-A2 patients. (See, e.g., Grey et al., Cancer Surveys 22:37-49 (1995).) This peptide can be used as antigen for HLA-A2+ patients (see, Central Data Analysis Committee “Allele Frequencies”, Section 6.3, Tsuji, K. et al. (eds.), Tokyo University Press, pp. 1066-1077). Similarly, peptides resembling other HLA binding motifs can be used.

In a typical embodiment, immature dendritic cells are cultured in vitro or ex vivo in the presence of a TLR agonist with or without Interferon γ and after a period of time the inflammation-activating lipid is added under conditions suitable for dendritic cell maturation, as described above. The predetermined antigen can be added prior to, together with, of subsequent to the TLR agonist also under suitable maturation conditions.

Optionally, the immature dendritic cells can be admixed with the predetermined antigen in a typical dendritic cell culture media without GM-CSF and IL-4. Following at least about 10 minutes to 2 days of culture with the antigen, the antigen can be removed, and the culture media supplemented with the TLR agonist with or without Interferon γ. The inflammation-activating lipid can be added after an appropriate maturation period. For example, the immature dendritic cells can be matured for at least 3 hours before the inflammation-activating lipid is added. The time prior to the addition of the inflammation-activating lipid can be 4 hours, 5 hours, 6 hours, 7 hours, 8 hours, 10 hours, and up to at least 24 hours. Cytokines (e.g., GM-CSF and IL-4) can also be added to the maturation media. Methods for contacting dendritic cells with antigen are generally known in the art. (See generally Steel and Nutman, J. Immunol. 160:351-360 (1998); Tao et al., J. Immunol. 158:4237-4244 (1997); Dozmorov and Miller, Cell. Immunol. 178:187-196 (1997); Inaba et al., J. Exp. Med. 166:182-194 (1987); Macatonia et al., J. Exp. Med. 169:1255-1264 (1989); De Bruijn et al., Eur. J Immunol. 22:3013-3020 (1992); the disclosures of which are incorporated by reference herein.)

The resulting mature, hyperactivated dendritic cells which present predetermined antigen can then be isolated, washed, and formulated for administration to an individual. In an optional embodiment the mature, hyperactivated, antigen presenting dendritic cells can be co-incubated in vitro or ex vivo with T cells, such as naïve T cells. T cells, or a subset of T cells, obtained from various lymphoid tissues for use as responder cells. Such tissues include but are not limited to spleen, lymph nodes, and/or peripheral blood. The cells can be co-cultured with mature, primed dendritic cells as a mixed T cell population or as a purified T cell subset. T cell purification or enrichment can be achieved by positive, or negative selection, including but not limited to, the use of antibodies directed to CD2, CD3, CD4, CD8, and the like.

By contacting T cells in vitro or ex vivo with mature, antigen presenting, hyperactivated dendritic cells, antigen-reactive, or activated, polarized T cells or T lymphocytes are provided. As used herein, the term “polarized” refers to T cells that produce high levels of IFNγ or are otherwise primed for inducing a type 1 (Th-1) response. Such methods typically include contacting immature dendritic cells with the bacteria, the bacterial component, or the bacterial lipopolysaccharide in combination with Interferon γ and the inflammation-activating lipid to prepare mature, hyperactivated, antigen presenting dendritic cells. In certain embodiments, the immature dendritic cells can be contacted in vitro or ex vivo with a predetermined antigen simultaneously with, prior to, of subsequent maturation under the appropriate conditions.

The immature dendritic cells can be co-cultured in vitro or ex vivo with T cells (e.g., naïve T cells) during maturation, or co-cultured with T cells (e.g., naïve T cells) after maturation and hyperactivation of the dendritic cells for inducing an enhanced type 1 response. The immature dendritic cells or mature dendritic cells can be further enriched prior to maturation. In addition, T cells can be enriched from a population of lymphocytes prior to contacting with the dendritic cells. In a specific embodiment, an enriched or purified population of CD4⁺ or CD8⁺ T cells is contacted with the dendritic cells. Co-culturing of the mature, primed dendritic cells with T cells leads to the stimulation of specific T cells which mature into antigen-reactive CD4⁺ T cells or antigen-reactive CD8⁺ T cells.

In another aspect, methods are provided for re-stimulation of T cells in vitro, by culturing the cells in the presence of mature hyperactive dendritic cells primed toward inducing a type 1 (Th1) T cell response. Such T cells optionally can be cultured on feeder cells. The mature, primed dendritic cells optionally can be irradiated prior to contacting with the T cells. Suitable culture conditions can include one or more cytokines (e.g., purified IL-2, Concanavalin A-stimulated spleen cell supernatant, or interleukin 15 (IL-15)). In vitro or ex vivo re-stimulation of T cells by addition of immature dendritic cells, the TLR agonist with or without Interferon γ and the inflammation-activating lipid and the predetermined antigen can be used to promote expansion of the T cell populations.

A stable antigen-specific, polarized T cell culture or T cell line can be maintained in vitro for long periods of time by periodic re-stimulation. The T cell culture or T cell line thus created can be stored, and if preserved (e.g., by formulation with a cryopreservative and freezing) used to re-supply activated, polarized T cells at desired intervals for long term use.

In certain embodiments, activated CD8⁺ or CD4⁺ T cells can be generated according to the method of the present disclosure. Typically, mature, primed dendritic cells used to generate the antigen-reactive, polarized T cells are syngeneic to the subject to which they are to be administered ((e.g., are obtained from the subject). Alternatively, dendritic cells having the same HLA haplotype as the intended recipient subject can be prepared in vitro using non-cancerous cells (e.g., normal cells) from an HLA-matched donor. In a specific embodiment, antigen-reactive T cells, including cytotoxic T lymphocytes (CTL) and Th1 cells, are expanded in vitro as a source of cells for immunostimulation.

In Vivo Administration of Cell Populations

In another aspect of the disclosure, methods are provided for administration of cell populations described above. The cell populations can include, mature, hyperactivated dendritic cells or activated, polarized T cells, or a cell population containing such cells, to a subject in need of immunostimulation. Such cell populations can include both mature, primed dendritic cell populations and/or activated, polarized T cell populations. In certain embodiments, such methods are performed by obtaining dendritic cell precursors or immature dendritic cells, differentiating and maturing those cells in vitro or ex vivo in the presence of the TLR agonist with or without Interferon γ, the inflammation-activating lipid, and a predetermined antigen to form a mature hyperactivated antigen presenting dendritic cell population primed towards inducing a Th1 response. The immature dendritic cells can be contacted in vitro or ex vivo with antigen prior to, simultaneously with, or subsequent maturation under the appropriate conditions. Such mature, hyperactivated, antigen presenting dendritic cells can be isolated, and formulated for administered directly to a subject in need of immunostimulation.

In a related embodiment, the mature, primed dendritic cells can be contacted in vitro or ex vivo with lymphocytes from a subject to stimulate T cells within the lymphocyte population. The activated polarized lymphocytes optionally followed by clonal expansion in cell culture of antigen reactive CD4⁺ and/or CD8⁺ T cells, can be administered to a subject in need of immunostimulation. In certain embodiments, activated, polarized T cells are autologous to the subject.

In another embodiment, the dendritic cells, T cells, and the recipient subject have the same MHC (HLA) haplotype. Methods of determining the HLA haplotype of a subject are known in the art. In a related embodiment, the dendritic cells and/or T cells are allogenic to the recipient subject. For example, the dendritic cells can be allogenic to the T cells and the recipient, which have the same MHC (HLA) haplotype. The allogenic cells are typically matched for at least one MHC allele (e.g., sharing at least one but not all MHC alleles). In a less typical embodiment, the dendritic cells, T cells and the recipient subject are all allogeneic with respect to each other, but all have at least one common MHC allele in common.

According to one embodiment, the T cells are obtained from the same subject from which the immature dendritic cells were obtained. After maturation and polarization in vitro or ex vivo, the autologous T cells are administered to the subject to provoke and/or augment an existing immune response. For example, T cells can be administered, by intravenous infusion, for example, at doses of about 10⁸-10⁹ cells/m² of body surface area (See, e.g., Ridell et al., Science 257:238-241 (1992), incorporated herein by reference). Infusion can be repeated at desired intervals, for example, monthly. Recipients can be monitored during and after T cell infusions for any evidence of adverse effects.

In another aspect of the disclosure, the immature dendritic cells can be matured by any standard method well known in the art including methods that contact immature dendritic cells with a new, clean, unused tissue culture substrate without the presence of a dendritic cell maturation agent. During maturation the maturing dendritic cells can be contacted with a predetermined antigen, such as a tumor specific or tumor associated antigen. Once matured, the dendritic cells can be formulated for administration to an individual and on administration the matured dendritic cell composition is co-administered with the inflammation-activating lipid.

The hyperactivated mature dendritic cells and activated T cells as produced herein can be formulated with physiologically acceptable carriers, excipients, buffers and/or diluents using methods and compositions well known to the skilled artisan. The dendritic cells or T cell compositions can be administered to a subject in need of immunostimulation. Typically, about 10² to about 10¹⁰ cells are suspended in a pharmaceutically acceptable carrier.

The matured dendritic cells and the inflammation-activating lipid can be administered in the same formulation or can be administered in separate compositions. When administered in separate compositions, the matured dendritic cells and the inflammation-activating lipid can be administered simultaneously or sequentially in either order. Typically, the mature or maturing dendritic cells will not be contacted with the inflammation-activating lipid until at least 3 hours after immature dendritic cell contact with the TLR agonist with or without IFNγ. In certain embodiments, the inflammation-activating lipid is administered topically to an area where the matured dendritic cells are administered, for example, by injection.

Treating subjects with the compositions of the present disclosure includes delivering therapeutically effective amounts. Therapeutically effective amounts include those that provide effective amounts, prophylactic treatments, and/or therapeutic treatments.

An “effective amount” is the number of cells necessary to result in a desired physiological change in a subject. Effective amounts are often administered for research purposes. Effective amounts disclosed herein do one or more of: (i) induce a Th1 immune response that is capable of reducing tumor or bacterial cell or viral proliferation or inducing tumor bacteria cell death or virus destruction and (ii) have an anti-cancer or anti-infective effect.

A “prophylactic treatment” includes an administration of a composition described herein to a subject who does not display signs or symptoms of a tumor to be treated or displays only early signs or symptoms of a tumor to be treated such that administration of a disclosed composition is for the purpose of diminishing, preventing, or decreasing the risk of developing a tumor or infection. Thus, a prophylactic treatment functions as a preventative treatment against a condition.

A “therapeutic treatment” includes a composition described herein is administered to a subject who displays symptoms or signs of a condition and is administered to the subject for the purpose of reducing the severity or progression of the condition.

The actual dose amount administered to a particular subject and/or the number of doses given, can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges and/or number of doses.

Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or even greater than 10¹¹.

As indicated, the compositions and formulations disclosed herein can be administered by, for example, injection, infusion, perfusion, or lavage and can more particularly include administration through one or more bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous infusions and/or bolus injections.

Administration of Maturing Hyperactive Dendritic Cells

According to another embodiment, dendritic cells can be induced to begin maturation in vitro or ex vivo with a dendritic cell maturation agent, such as, for example, the TLR agonist with or without Interferon γ and the inflammation-activating lipid according to the present disclosure. In this embodiment, the dendritic cells are not allowed to fully mature prior to administration. These dendritic cells like immature dendritic cells retain the ability to uptake and process antigen and like fully mature dendritic cells can induce an enhanced anti-tumor response subsequent to administration to an individual.

Fully mature dendritic cells differ qualitatively and quantitatively from immature DCs. Once fully mature, DCs express higher levels of MHC class I and class II antigens, and higher levels of T cell co-stimulatory molecules, such as CD80 and CD86. These changes increase the capacity of the dendritic cells to activate T cells, for example, by increasing the antigen density on the cell surface, as well as the T cell activation signal through the counterparts of the co-stimulatory molecules on the T cells, e.g., CD28 and the like. In addition, mature DCs produce large amounts of cytokines, which stimulate and polarize the T cell response. These cytokines include interleukin 12 associated with a Th1-type immune response and interleukin-10 and interleukin-4 associated with a Th2-type immune response. Typically, the inflammation-activating lipid will be administered together with or after administration of the partially mature dendritic cells.

Generally, methods for ex vivo DC generation as disclosed above comprise obtaining a cell population enriched for DC precursor cells from a subject and then differentiating the DC precursor cells in vitro or ex vivo to form immature dendritic cells and inducing the immature dendritic cells to mature into fully mature DCs. Typically, during this process the maturing DCs are contacted with a predetermined antigen in vitro or ex vivo for uptake and processing as the DCs become mature. Some believe that the DCs must be terminally differentiated, or they will de-differentiate back into monocytes/macrophages and lose much of their immune-potentiating ability. Ex vivo maturation of DCs generated from monocytes has been successfully accomplished with methods and agents well known in the art and further described above. In the present embodiment, maturing dendritic cells are preferred as the maturing dendritic cells are administered to the patient and take up and process antigen in vivo while fully maturing and transiting to a lymph node for contact with naïve T cells subsequent to administration.

The maturing dendritic cells as produced herein can be formulated with physiologically acceptable carriers, excipients, buffers and/or diluents using methods and compositions well known to the skilled artisan. The maturing dendritic cells can be administered directly to a subject in need of immunostimulation. Typically, about 10² to about 10¹⁰ cells are suspended in a pharmaceutically acceptable carrier. The cells are injected either into the tumor directly or into a region near to, adjacent to, or in circulatory or lymphatic contact with the tumor or tumor bed to ensure that the cells have access to the tumor antigens.

For example, but not by limitation, the cells can be administered directly into a tumor, into the tumor bed subsequent to surgical removal or resection of the tumor, peritumoraly, into a draining lymph node in direct contact with the tumor, into a blood vessel or lymph duct leading into, or feeding a tumor or organ afflicted by the tumor, e.g., the portal vein or a pulmonary vein or artery, and the like. The administration of the maturing dendritic cells can be either simultaneous with or subsequent to other treatments for the tumor, such as administration of the inflammation-activating lipid when it is administered as a separate composition, chemotherapy or radiation therapy. Further, the maturing dendritic cells can be co-administered with another agent, which agent acts as an adjuvant to the maturation of the dendritic cell and/or the processing of antigen within the tumor or region near or adjacent to the tumor, such as the inflammation-activating lipid. In addition, the dendritic cells can also be formulated or compounded into a slow release matrix for implantation into a region in or around the tumor or tumor bed such that cells are slowly released into the tumor, or tumor bed, for contact with the tumor antigens.

The maturing dendritic cells can be administered by any means appropriate for the formulation and mode of administration. For example, the cells can be combined with a pharmaceutically acceptable carrier and administered with a syringe, a catheter, a cannula, and the like. As above, the cells can be formulated in a slow release matrix. When administered in this fashion, the formulation can be administered by a means appropriate for the matrix used. Other methods and modes of administration applicable to the present disclosure are well known to the skilled artisan.

In another embodiment, the maturing dendritic cells and the recipient subject have the same MHC (HLA) haplotype. Methods of determining the HLA haplotype of a subject are known in the art. In a related embodiment, the maturing dendritic cells are allogeneic to the recipient subject. The allogeneic cells are typically matched for at least one MHC allele (e.g., sharing at least one but not all MHC alleles). In a less typical embodiment, the maturing dendritic cells and the recipient subject are all allogeneic with respect to each other, but all have at least one MHC allele in common.

In one embodiment the maturing dendritic cells produced by any one of the above methods can be administered as an adjuvant to radiation therapy, chemotherapy, or a combination thereof. For example, the maturing hyperactivated dendritic cells can be administered prior to, simultaneous with, or subsequent to radiation therapy, chemotherapy, or a combination thereof.

In another embodiment a method is provided for producing an enhanced anti-tumor immune response and/or clinical response comprising administrating a composition comprising a cell population enriched for human dendritic cells have been induced to begin maturation and are hyperactivated in vitro with a TLR agonist with or without Interferon γ (IFNγ) and the inflammation-activating lipid. The maturing hyperactivated dendritic cells are isolated and combined with a pharmaceutically acceptable carrier; wherein the composition is administered into a tumor, a tumor bed or a tissue area surrounding a tumor in an individual in need of such treatment.

In another embodiment the immature dendritic cells are induced to begin maturation with a dendritic cell maturation agent, for example, heat inactivated BCG, in combination with IFNγ. Prior to full and complete maturation, the maturing dendritic cells are isolated and formulated for administration to an individual. The formulation can comprise the inflammation-activating lipid or the inflammation-activating lipid can be a separate composition that is co-administered with the composition comprising the maturing dendritic cells. The composition comprising the inflammation-activating lipid can be administered before, concurrently, or subsequent to the administration of the maturing dendritic cells. In this embodiment, the inflammation-activating lipid is acting as an adjuvant to the maturing dendritic cells subsequent to their administration to an individual. The antigen is taken up by the activated maturing dendritic cells in vivo or ex vivo subsequent to administration and processed for presentation to T cells in the body of the individual. As above, the activated maturing dendritic cells and the inflammation-activating lipid can be administered into a tumor, a tumor bed or a tissue area surrounding a tumor in an individual in need of such treatment.

In yet another embodiment, immature dendritic cells can be matured together with a predetermined antigen by any method known in the art. For example, the immature dendritic cells can be matured with a combination of a TLR agonist such as BCG and IFNγ together with a tumor lysate. The maturing dendritic cells uptake and process tumor antigen during maturation, which antigen can be displayed to naïve T cells on administration to the individual from whom the tumor was resected. Once fully mature, the dendritic cells now presenting antigen are isolated and formulated for administration to the individual. The formulated mature dendritic cells can be administered together with the inflammation-activating lipid, for example, oxPAPC, to induce an enhanced tumor specific Th1 immune response as compared to the formulated mature dendritic cells when administered alone. In this embodiment the inflammation-activating lipid can be formulated together with the mature dendritic cells or can be formulated in a separate composition. When formulated as a separate composition the formulated mature dendritic cells and the inflammation-activating lipid can be administered simultaneously or sequentially in any order. In certain embodiments the inflammation-activating lipid can be administered as a topical composition to the area or the skin or mucosa where the formulated mature dendritic cells are administered. The combination of the formulated mature dendritic cells and the inflammation-activating lipid can induce an enhance antigen specific immune response to the predetermined antigen as compared with the formulated mature dendritic cells when administered alone.

All the compositions described above, including hyperactive mature dendritic cell compositions, maturing dendritic cell compositions, and activated T cell compositions, and the like, can be used in combination with any other method to treat a tumor, or for example, the methods and compositions of the present disclosure can be used in combination with surgical resection of a tumor, chemotherapy (cytotoxic drugs, apoptotic agents, antibodies, and the like), radiation therapy, cryotherapy, brachytherapy, immune therapy (administration of antigen specific mature activated dendritic cells, NK cells, antibodies specific for tumor antigens, and the like), and the like. All the above methods can also be used in any combination. Combination treatments can be concurrent or sequential and can be administered in any order as determined by the treating physician.

### EXAMPLES

The following examples are provided merely as illustrative of various aspects of the compositions and methods described herein and shall not be construed to limit the disclosure in any way.

### Example 1: Differentiation and Maturation of Monocytic Dendritic Cell Precursors

In a first study, a cell population enriched for human monocytic dendritic cell precursors is induced to differentiate in vitro or ex vivo into immature dendritic cells by contact with a dendritic cell differentiation agent and which are in turn induced to mature by the contacting of the precursors with a dendritic cell maturation agent and an inflammation-activating lipid as defined above. In this example, the dendritic cell differentiation agent is a combination of GM-CSF and 2% human serum albumin. Similar results, differentiation of the monocytic dendritic cell precursors into immature dendritic cells, can be achieved using GM-CSF in combination with IL-4, IL-7, IL-13 or IL-15. After dendritic cell differentiation the enriched population of immature dendritic cells is induced to mature and is hyperactivated with a dendritic cell maturation agent and a dendritic cell hyperactivation agent (an inflammation-activating lipid), wherein the dendritic cell maturation agent is an agonist of a Toll-like Receptor. In this particular example, a Toll-like Receptor 4 (TLR4) agonist LPS is used and can also comprise a bacteria, bacterial product or component of LPS, or another bacterial LPS. The maturation of the dendritic cells is biased to induce of Th1 response with the addition of Interferon γ (IFNγ). Hyperactivation is induced with an inflammation-activating lipid such as oxPAPC.

Specifically, an enriched cell population of human monocytic dendritic cell precursors are cultured with GM-CSF with 2% human serum albumin to produce a cell population enriched for immature dendritic cells. The enriched population of immature dendritic cells are induced to begin maturation and activation with LPS and the inflammatory-activating lipid is added either simultaneously or after from 1 to 12 hours to induce full maturation and hyperactivation. The status of the mature dendritic cells can be ascertained by determining the production of, for example, IL-12, TNFα, and/or IL-1β. In addition, the ability of the mature hyperactivated dendritic cells to induce the production of effector and memory T cells, or on the terminal differentiation and/or activation of CD4⁺ and CD8⁺ T cells can be determined by known methods.

In another embodiment monocytic dendritic cell precursors were cultured in cell culture media supplemented with GM-CSF and IL-4 to induce differentiation to form immature dendritic cells and subsequently the immature dendritic cells are cultured in a combination of a TLR agonist, such as BCG or R848, and with or without IFNγ for a time period sufficient to fully mature the dendritic cells. Hyperactivation is induced by contacting the maturing and activating dendritic cells with an inflammation-activating lipid simultaneously or sequentially with the dendritic cell maturation agent/TLR agonist. The inflammation-activating lipid can be added to the culture at the same time as the dendritic cell maturation agent or from 1 to 20 hours, or more subsequent to the initiation of maturation.

In one embodiment, a predetermined antigen is contacted with the immature dendritic cells prior to, simultaneously with, or subsequent to contacting the immature dendritic cells with a TLR agonist, such as a bacterial LPS, R848, or another dendritic cell maturation disclosed herein. The TLR agonist can be combined with IFNγ and also can be used with IFNγ in combination with or without the inflammation-activating lipid. More specifically, a tumor cell lysate is added to the culture during the period of dendritic cell differentiation and/or maturation. If added subsequent to maturation, known methods, such as, for example, osmatic loading can be used to enhance antigen uptake.

In an optional embodiment, the monocytic dendritic cell precursors are contacted only with a dendritic cell maturation agent and IFNγ, no inflammation-activating lipid was included. Instead. the mature dendritic cells when fully matured are formulated with a pharmaceutically acceptable carrier and administered together with the inflammation-activating lipid. The inflammation-activating lipid can be combined in a single formulation with the mature dendritic cells, or separately. Optionally, the inflammation-activating lipid is formulated as a separate composition and either administered prior to, simultaneously with, or subsequent to administration of the mature dendritic cells.

The amount of IL-12, IL-2, TNFα and IL-1β, or other inflammatory cytokine can be determined by use of a commercially available test, such as an ELISA or a multiplex assay. The terminal differentiation of CD4⁺ and CD8⁺ T cells and the level of effector and memory T cells can be determined by methods well known in the art.

### Example 2: Production and Maturation of Hyperactive Dendritic Cells

In this example, a cell population enriched immature dendritic cells is obtained by means well known in the art. The enriched cell population of immature dendritic cells are cultured together with a combination of BCG, or R848, with or without IFNγ and oxPAPC for 12 to 24 hours, or until the immature dendritic cells are fully mature. The fully mature dendritic cells are then assayed for production of IL-2, IL-12 p70, TNFα and/or IL-1β. In addition, the ability of the fully matured dendritic cells to induce terminal differentiation and/or activation of CD4⁺ and CD8⁺ T cells and the level of effector and memory T cells is determined by methods well known in the art.

In a separate study, tumor cell lysate is added to the maturing dendritic cells for uptake and processing. The maturing dendritic cells are cultured until they are fully mature and then samples of the mature hyperactivated dendritic cells are tested as above.

In addition, the fully mature dendritic cells are also isolated and washed with, for example PBS or fresh culture media. The isolated hyperactivated dendritic cells can then be formulated for administration or cryopreserved for later use.

### Example 3: Generation of Hyperactive Dendritic Cells In Vitro from Human Peripheral Blood

In this example, dendritic cells (DCs) were generated in vitro from the PBMCs of healthy human donors, and the DCs were induced to mature and hyperactivate with an agonist of a Toll-like receptor (TLR) and an inflammation-activating lipid to generate hyperactive mature dendritic cells. The inflammation-activating lipid used in this example was oxidized 1-palmitoyl-2-arachidonyl-sn-glycero-3-phosphorylcholine (oxPAPC). Four TLR agonists were tested in this example including: bacterial lipopolysaccharide (LPS) (TLR4), the synthetic diacylated lipopeptide Pam2CSK4 (Pam2; TLR2/TLR6), the synthetic triacylated lipopeptide Pam3CSK4 (Pam3; TLR2/TLR1), and the synthetic double-stranded RNA analog polyinosinic-polycytidylic acid (Poly I:C) (TLR3). The activation of DCs with the various TLR agonists was followed by stimulation with the inflammation-activation lipid being tested to determine which lead to hyperactivation of these cells. Those dendritic cells that are hyperactivated are expected to be more effective in treating tumor-bearing animals and humans.

TLRs are a class of pattern-recognition receptors (PRRs), which detect microbial molecules classically referred to as pathogen associated molecular patterns (PAMPs). The TLR immune receptors are expressed on the membranes of leukocytes, including dendritic cells, and the binding of TLR agonists triggers molecular events that can lead to immune responses and antigen-specific acquired immunity. Bacterial lipopolysaccharide (LPS) is a strong agonist of TLR4 with the ability to enhance immune responses to soluble antigens. LPS stimulates immune system cells by the TLR4 pathway, which recognizes common PAMPs. Pam2 is a synthetic lipopeptide (LP). Such lipopeptides mimic bacterial lipoproteins which are strong immune modulators that activate early innate host responses after infection. Pam2 is capable of inducing signaling through the TLR2 and TLR6 pathways and can also do so in a TLR6-independent manner. Pam3 is another synthetic LP and is a TLR2/TLR1 ligand. Poly I:C is a synthetic analog of double-stranded RNA, a molecular pattern associated with viral infections. Poly I:C is recognized by TLR3.

### Methods

DCs derived from cryopreserved PBMCs (Hemacare, Los Angeles, CA) were plated and distributed evenly in all wells of a 6-well tissue culture plate in phenol red free RPMI-1640/10% human heat-inactivated AB serum supplemented with 500 U/ml of recombinant human GM-CSF and 500 U/ml of recombinant human IL-4. Additional culture medium supplemented with GM-CSF/IL4 was added two days after the initiation of the culture. On day 8, immature DCs were harvested (suspension, adherent cells and non-adherent fractions) and plated in triplicate at 2×10⁵ cells/well in a 96-well flat bottom tissue culture plate. Samples of the immature DCs were stimulated with 1 μg/ml of LPS (Sigma), 1 μg/ml of Pam2 (Invivogen), 1 μg/ml of Pam3 (Invivogen), or 25 μg/ml of Poly I:C HMW (Invivogen) for 3 hrs. Titrated concentrations of oxPAPC (Invivogen)—at 1 μg/ml, 3 μg/ml, 10 μg/ml, 30 μg/ml, and 90 μg/ml—were added to appropriate samples and the cells were cultured for an additional 18 hours. Cold PBS incubation was performed if DC stimulation resulted in activated DCs that were too tightly adherent for proper harvest.

DCs were stained and flow cytometry analysis was performed to assess the expression of the following dendritic cell surface markers: CD1c, CD11b, CD40, CD80, CD86, CD123, CD141, MHC-I, MHC-II, and for the percentage of Live or Dead cells.

Cytokine production in 100 μl of culture medium was assessed by a mouse cytokine and chemokine panel, across 34 protein targets (Eotaxin/CCL11; GM-CSF; GROα/CXCL1; conIFNα; IFNγ; IL1β; IL1α; IL1RA; IL2; IL4; IL5; IL6; IL7; IL8/CXCL8; IL9; IL10; IL12 p70; IL13; IL15; IL17A; IL18; IL21; IL22; IL23; IL27; IL31; IP10/CXCL10; MCP1/CCL2; MIP1α/CCL3; MIP1β/CCL4; RANTES/CCL5; SDF1α/CXCL12; TNFα; TNFβ/LTA), using a Luminex immunoassay system using the manufacturer's instructions. The Luminex system can simultaneously detect many targets in a single sample.

### Discussion

It was anticipated based on our observations that human DCs matured with certain TLR agonists and hyperactivated with the inflammation-activating lipid oxPAPC would promote production of cytokines that can induce a Th1 response, leading to an increase in effector (e.g., CD8⁺ cytotoxic T-cells) and memory T-cells.

In this example, introduction of oxPAPC at increasing concentrations to TLR-treated immature dendritic cells resulted in enhanced expression of cell surface markers typically associated with DC maturation, such as CD69 and CD83, and costimulatory molecules like CD86 and MHC-II, which would result in enhanced T-cell stimulation. Cytokine analysis showed modulation of IL-10 to lower concentrations at higher concentrations of oxPAPC for most TLRs, with some coincident modulation of IL-5 and IL-27 production (e.g., LPS). IL-10 reduction can prevent the induction of regulatory T cells, permitting a stronger Th1 immune response to occur.

### Example 4: In Vitro Study of Cytokine Production by Murine Bone Marrow Derived Dendritic Cells

### Background

In this example, immature dendritic cells (DCs) were generated in vitro from bone marrow derived precursors in C57BL/6 and BALB/c mice. The enriched population of immature dendritic cells was induced to mature and activate with an inflammation-activating lipid and an agonist of a Toll-like receptor (TLR) to generate hyperactive mature dendritic cells. As in the previous example, the same four TLR agonists: LPS, Pam2, Pam3, and Poly I:C were used. The inflammation-activating lipid oxPAPC was used to hyperactivate the maturing dendritic cells. It was expected that activation of murine bone marrow-derived DCs followed by stimulation with inflammation-activating lipids would lead to hyperactivation of these cells, and that such hyperactivated DCs would be more effective in treating tumor-bearing animals and humans.

### Methods

Bone marrow cells were isolated from femurs and tibias of C57BL/6 and Balb/c mice. Lineage negative progenitors were enriched using the Lineage Cell Depletion Kit (Miltenyi). Dendritic cell precursors were plated and distributed evenly in all wells of a 6 well tissue culture (TC) plate in RPMI 1640/10% fetal bovine serum (FBS)/penicillin/streptomycin/L-glutamine/2-mercaptoethanol (2-ME) supplemented with GM-CSF (10 ng/ml) and IL-4 (10 ng/ml) in 3 mls/well. On day 2 of culture, non-adherent cells were harvested and re-plated into a new plate. Additional culture medium supplemented with GM-CSF/IL-4 was added on days 4 and 6 of culture. On day 8, immature DCs were harvested (suspension and loosely adherent cells only) and plated in triplicate at 2×10⁵ cells/well in a 96-well flat bottom TC plate. DCs were stimulated with 1 μg/ml of LPS (Sigma), 1 μg/ml of Pam2 (Invivogen), 1 μg/ml of Pam3 (Invivogen), or 1 μg/ml of Poly I:C HMW (Invivogen) for 3 hrs. Titrated concentrations of oxPAPC (Invivogen)—at 1 μg/ml, 3 μg/ml, 10 μg/ml, 30 μg/ml, and 90 μg/ml—were then added to the appropriate samples and culture was continued for an additional 18 hours. Cold PBS incubation was performed if DC stimulation resulted in activated DCs that were too tightly adherent for proper harvest.

Flow cytometry analysis was performed to assess the expression of dendritic cell surface markers (CD11c, CD11b, CD80, CD83, CD69, CD86, CD40, MHC-I, MHC-II, CD205, and the percentage of Live/Dead cells was determined). The mature hyperactivated dendritic cells were expected to show increases in, for example, CD80, CD86, CD83, CD40, MHC-1 and/or MHC-II.

Cytokine production in 100 μl of culture medium was assessed by a mouse cytokine and chemokine panel, across 26 protein targets (IFNγ; IL12 p70; IL13; IL1β; IL2; IL4; IL5; IL6; TNFα; GM-CSF; IL18; IL10; IL17A; IL22; IL23; IL27; IL9; GROα; IP10; MCP1; MCP3; MIP1α; MIP1β; MIP2; RANTES; and Eotaxin), using Luminex xMAP® technology, an immunoassay system that can simultaneously detect many targets in a single sample. It was expected that stimulation with oxPAPC and TLR agonists would upregulate at least a number of inflammatory cytokines including, for example, IL10, TNFα, IL-2 and/or IL12 p70.

### Discussion

It was anticipated that in this example DCs of murine origin matured with TLR agonists and hyperactivated with the inflammation-activating lipid oxPAPC would produce cytokines that induce a Th1 response, ultimately leading to an adaptive immune response that upregulates effector (e.g., CD8⁺ cytotoxic T-cells) and memory T-cells.

In this example, increasing the concentration of oxPAPC resulted in some upregulation of IL-12 p70 (in LPS, Pam2, and Pam3 treated groups). There was also some modulation of IL-13 production at higher oxPAPC concentrations, especially in the LPS and Pam3 treated groups, which also showed some downregulation of IL-10. The downregulation of IL-10 typically results in suppression of a Th2 response, which, in combination with increased production of IL-12 p70, can indicate favorable conditions for immune therapeutic treatments.

### Example 5: In Vitro Study of Cryopreserved Enriched Murine Immature DCs

In this example, cryopreserved C57BL/6 and BALB/c immature DCs were stimulated in vitro with agonists of Toll-like receptors (TLR) and inflammation-activating lipids to generate hyperactive mature dendritic cells. The immature DCs were stimulated with four TLR agonists including bacterial lipopolysaccharide (LPS; TLR4), heat-inactivated Bacillus Calmette-Guérin (hBCG; TLR2/TLR4), polyinosinic-polycytidylic acid (Poly I:C; TLR3), and 1-(4-Amino-2-(ethoxymethyl)-1H-imidazo[4,5-c]quinolin-1-yl)-2-methylpropan-2-ol (R-848; TLR7/TLR8). As in the previous example, Pam 2 (TLR2/TLR6) and Pam3 (TLR2/TLR1) were included untreated and at the highest inflammation-activating lipid concentration only, for comparison across studies. Also, as in the previous example the inflammation-activating lipid oxPAPC was used, and in addition 1-palmitoyl-2-glutaryl phosphatidylcholine (PGPC; Cayman), a component derived from oxPAPC was included. The activation of murine bone marrow-derived immature DCs with TLR agonists followed by stimulation with inflammation-activating lipids was expected to lead to hyperactivation of these cells, and that such hyperactivated DCs will be more effective in treating tumor-bearing animals and humans.

### Methods

Immature cryopreserved C57BL/6 and BALB/c DCs (Cellero) were rapidly thawed in a water bath at 37° C. and the cells were resuspended in 10 times the volume of RPMI 1640/10% FBS/penicillin/streptomycin/L-glutamine/2-ME. Cells were washed twice using complete media.

Thawed cells were plated in triplicate at 2×10⁵ cells/well in a 96-well flat bottom TC plate. DCs were primed with 1 μg/ml of LPS (Sigma Aldrich), 1:400 dilution of hBCG (Organon Teknika/Cognate), 1 μg/ml of R848 (Sigma Aldrich), 1 μg/ml Pam2 (Invivogen), 1 μg/ml Pam3 (Invivogen), and 25 μg/ml of Poly I:C HMW (“high molecular weight”; Invivogen) for 3 hrs. Titrated concentrations of oxPAPC (Invivogen) or PGPC (Cayman)—at 1 μg/ml, 3 μg/ml, 10 μg/ml, 30 μg/ml, and 90 μg/ml—were added to appropriate samples and cultured for an additional 48 hours.

If the activated and matured DCs were tightly adherent following stimulation with the TLRs and inflammation-activating lipids, the supernatant with the non-adherent cells was removed and this suspension was put on ice. Cold PBS was added to the wells and the plate was placed in the refrigerator for 30 minutes to allow the adherent cells to release. Adherent and non-adherent cells were collected with the PBS and added to the previously harvested cells kept on ice. The cells were washed once with HBSS or DPBS prior to flow cytometric phenotyping, with triplicate wells combined for flow cytometric analysis.

DCs were stained with labeled monoclonal antibodies and flow cytometry analysis was performed to assess expression of the following dendritic cell surface markers: CD11b, CD11c, CD14, CD40, CD69, CD80, CD83, CD86, CD205, MHCI, MHCII, and to determine if the cells were alive or dead.

Cytokine production in 100 μl of culture medium was assessed by a mouse cytokine and chemokine panel, across 26 protein targets (IFNγ; IL12 p70; IL13; IL1β; IL2; IL4; IL5; IL6; TNFα; GM-CSF; IL18; IL10; IL17A; IL22; IL23; IL27; IL9; GROα; IP10; MCP1; MCP3; MIP1α; MIP1β; MIP2; RANTES; and Eotaxin), using Luminex xMAP® multiplex technology using the manufacturer's. The xMAP system is an immunoassay system that can simultaneously detect many targets in a single sample.

### Discussion

In this example immature murine DCs were matured with TLR agonists and hyperactivated with the inflammation-activating lipid oxPAPC or its component lipid, PGPC, looking to see if these lipids would enhance cytokine production toward a Th1 response, as before. Stimulated DCs treated with increasing concentrations of both oxPAPC and PGPC upregulated production of IL-1β. This increase in IL-1β is a hallmark of DC hyperactivation.

### Example 6: In Vitro Study on the Differentiation, Maturation, and Hyperactivation of Cryopreserved Human Dendritic Cell Precursors

In this example, an enriched cell population of immature DCs was generated in vitro using frozen human PBMCs. The obtained cell population of enriched human immature DCs was activated and matured with agonists of various Toll-like receptors (TLRs) and subsequently further activated with an inflammation-activating lipid to generate hyperactive mature dendritic cells. The cell population enriched for immature DCs was induced to mature and activate with four of the TLR agonists used in Example 5, i.e., LPS, heat inactivated BCG, Poly I:C, and R848. Also, as in Example 5, the DCs were further stimulated with the inflammation-activating lipids oxPAPC and PGPC.

### Methods

Frozen human PBMCs (Hemacare, Los Angeles, CA) were thawed and washed, and then plated in T75 TC flasks (approximately 2×10⁶ cells/ml) for at least 2 hours at 37° C. After adherence of monocytes to the tissue culture substrate surface, non-adherent cells were removed, and the flasks were gently rinsed with warm complete medium. Adherent monocytes were cultured in phenol red free RPMI-1640/10% human heat-inactivated AB serum supplemented with 500 U/ml of recombinant human GM-CSF and 500 U/ml of recombinant human IL-4 for 7 days. Culture medium was supplemented with GM-CSF/IL-4 two days after initiation of culture.

On day 8, an enriched population of immature DCs was harvested by collection of the suspension, adherent cells and non-adherent fractions, and the cells were plated in triplicate at 2×10⁵ cells/well in a 96-well flat bottom TC plate. The enriched immature DCs were stimulated with the various TLRs: 1 μg/ml of LPS (Sigma Aldrich), 25 g/ml of Poly I:C HMW (“high molecular weight”; Invivogen), a 1:400 dilution of heat-inactivated BCG (Organon Teknika/Cognate) and 1 μg/ml of R848 (Sigma Aldrich) for 3 hrs. Titrated concentrations of inflammation-activating lipids oxPAPC (Invivogen) or PGPC (Cayman)—at 1 μg/ml, 3 μg/ml, 10 μg/ml, 30 μg/ml, and 90 μg/ml were added to the appropriate samples and cultured for an additional 18 hours.

Following stimulation with the TLRs and inflammation-activating lipids, if the activated DCs were tightly adherent to the tissue culture substrate surface, the supernatant with the non-adherent cells was removed and the suspension was put on ice. Cold PBS was added to the wells and the plate was put into a refrigerator for 30 minutes to allow the adherent cells to release from the tissue culture substrate. The collected cells in PBS were collected and added to the previously harvested cells that were kept on ice. The collected cells were washed once with HBSS or DPBS prior to triplicate well combination in preparation for flow cytometric phenotyping and analysis.

DCs were stained and flow cytometry analysis was performed to assess expression of the following dendritic cell surface markers: CD1c, CD11c, CD40, CD69, CD80, CD83, CD86, CD123, CD141, MHC-I, MHC-II, and for Live and Dead cells.

As above, cytokine production in 100 μl of culture medium was assessed by a mouse cytokine and chemokine panel, across 34 protein targets (Eotaxin/CCL11; GM-CSF; GROα/CXCL1; IFNα; IFNγ; IL13; IL1α; IL1 RA; IL2; IL4; IL5; IL6; IL7; IL8/CXCL8; IL9; IL10; IL12 p70; IL13; IL15; IL17A; IL18; IL21; IL22; IL23; IL27; IL31; IP10/CXCL10; MCP1/CCL2; MIP1α/CCL3; MIP1D/CCL4; RANTES/CCL5; SDF1α/CXCL12; TNFα; TNFβ/LTA), using an Luminex xMAP® immunoassay system as instructed by the manufacturer. The Luminex xMAP® system can simultaneously detect many targets in a single sample.

### Discussion

DCs of human origin matured with TLR agonists and hyperactivated with the inflammation-activating lipid oxPAPC, as well as its component lipid, PGPC, would promote cytokine production that helps induce a Th1 response, leading to an increase in effector (e.g., CD8⁺ cytotoxic T-cells) and memory T-cells. Cell surface markers typically associated with DC maturation and costimulatory molecules that enhance T-cell stimulation would be upregulated in this environment. Production of IL-1 family cytokines (including IL-1β) and IL-12 would ensue, coincident with IL-10 suppression, creating a favorable environment for a T-cell mediated adaptive immune response.

### Example 7: In Vivo Studies of Syngeneic Tumor Cells—CT26, 4T1, Madison 109, and A20 with Administration of Hyperactivated Dendritic Cells

In this example, tumor lysate-loaded mature murine DCs produced ex vivo using various combinations of TLRs, plus IFNγ (in some cases), and an inflammation-activating lipid, to create hyperactivated DCs, were administered to syngeneic mice to determine which of the hyperactive DCs were more effective in slowing tumor growth in tumor-bearing animals. DCs were loaded with tumor cell lysate from various syngeneic BALB/c carcinoma model cell lines and further combined with a TLR, plus IFNγ, and an inflammation-activating lipid, to generate hyperactive DCs, which were systemically administered to female BALB/c mice. The inflammation-activating lipids used were again oxidized 1-palmitoyl-2-arachidonyl-sn-glycero-3-phosphorylcholine (oxPAPC; Invivogen) and 1-palmitoyl-2-glutaryl phosphatidylcholine (PGPC; Cayman). 1-palmitoyl-2-(5-oxovaleroyl)-sn-glycero-3-phosphatidylcholine (POV-PC; Cayman), was also included in this study. PGPC and POVPC are components derived from oxPAPC.

The three TLR agonists used in this example to induced the maturation of the enriched immature dendritic cells were bacterial lipopolysaccharide (LPS; TLR4), polyinosinic-polycytidylic acid (Poly I:C; TLR3), and 1-(4-amino-2-(ethoxymethyl)-1H-imidazo[4,5-c]quinolin-1-yl)-2-methylpropan-2-ol (R848, Sigma Aldrich) were included. R848 is a potent synthetic agonist of TLR7/TLR8 and typically increases cytokine levels of TNF-α, IL-6 and IFN-α. Thus, among the three different TLRs included in this example, TLR3, TLR4, and TLR7/TLR8 were explored as pathways that could influence the course of successful DC maturation and hyperactivation inducing an enhanced immune response.

Four (4) different syngeneic carcinoma mouse model cell lines were selected as the source of tumor cell lysate for this example. Syngeneic mouse models utilize immunocompetent mice bearing tumors derived from the strain of origin. Immunogenic mouse models with available data describing growth kinetics and responses to standard of care agents (e.g., anti-CTLA4 and PD-1 checkpoint inhibitors) were selected because these models can provide a better source of comparison to approved agents.

To show that the methods and compositions of this disclosure have broad applicability to different cancers, this example included mouse models representing a diversity of tumor histotypes and response characteristics. The following four mouse models were selected: Madison 109 (lung carcinoma), CT26 (colon carcinoma), 4T-1 (breast carcinoma), and A20 (lymphoma). The CT26 and A20 models are “responsive” models, and the 4T-1 and Madison 109 models are “refractory” models. All these models are from and syngeneic with BALB/c mice.

### Preparation of Tumor Cell Lysate

At least 2×10⁹ tumor cells from each cell line (CT26, Madison 109, 4T-1, and A20) were harvested in log phase. Adherent cells were harvested with Versene (or equivalent) cell dissociation solution, with enzymatic cell dissociation methods avoided. Cells were rinsed twice with ice cold DPBS to remove serum from the cells.

Cells were pelleted at 400×g for 5 minutes after each wash. After centrifugation, the wash supernatant was removed and discarded. The tumor pellet was resuspended in an appropriate volume of RPMI-1640 without phenol red (at 150 to 300 million cells/ml) and transferred to microcentrifuge tubes. The cell suspension tubes were then frozen for longer than (>) one (1) hour at −80° C. and then thawed at room temperature. Alternatively, tumor pellets were snap-frozen in liquid nitrogen, and thawed rapidly (<5 minutes) in a 37° C. dry bath.

The freeze thaw process was repeated five (5) additional times for a total of six (6) freeze-thaw cycles to effectively lyse the tumor cells. Processing of the tumor cells was optionally performed over an extended period—up to several days—as a result of the ability to leave the material frozen during the freeze steps of the freeze-thaw process. If viscous, lysates were sonicated on ice for 30 seconds. Tubes containing thawed, lysed cell suspension were centrifuged at full speed for three (3) minutes on a tabletop centrifuge. The tumor lysate (supernatant) was pooled in a sterile 50 ml centrifuge tube and was sampled for protein concentration determination using a DC protein assay. Up to 2 mg/ml of total protein was adjusted and filtered through a 0.2 m filter. Filtered lysate was aliquoted into internally threaded, labeled cryovials, and capped tightly. A standard colorimetric protein assay (BioRad) was used to verify the final protein concentration of the filtered lysate. Tumor lysate for each tumor cell line was stored at −80° C. until ready for use.

Preparation of Dendritic Cell from Immature Dc as Positive Control

Immature DCs were incubated with tumor lysate medium comprised of tumor lysate, diluted at 100 μg/ml in RPMI-1640 supplemented with 10% FBS, 1% L-glutamine, 1% penicillin and streptomycin, 0.1% 0.1M 2-ME and 10 ng/ml of murine recombinant GM-CSF. DCs were resuspended at 1.0×10⁶ cells/ml to 1.5×10⁶ cells/ml. The tumor cell lysate medium cell suspension was then placed in a new culture flask labeled to reflect these additions and placed back in the incubator for fourteen (14) to twenty (20) hours. After this incubation period, adherent and non-adherent cells were harvested.

Supernatant consisting of non-adherent cells was removed, and the suspension was placed on ice. Cold PBS was added to the wells and the plate was placed in the refrigerator for 30 minutes to allow the adherent cells to release. Cell scrapers were discretionarily used to remove cells still stuck following cold PBS treatment. The cells with PBS were collected and added to the previously harvested cells on ice. Cells were washed once with RPMI (no additives) prior to resuspension for injection.

### Pulsing and Cryopreservation of DCs

Sufficient cryopreserved BALB/c DCs (Cellero) for each study in this example were thawed and rinsed twice with RPMI-1640 supplemented with 10% FBS, 1% L-glutamine, 1% penicillin and streptomycin and 0.1% 0.1 M 2-ME (the total number of cryopreservations and timing of the freezings varied among the lots). 10 ng/ml of murine recombinant GM-CSF were added freshly to the media to generate immature DCs. Cells were pelleted at 400×g for 4 minutes. Flow cytometric phenotypic analysis was performed, looking at % cells alive, as well as the following cell surface markers: CD11b, CD11c, CD14, CD40, CD69, CD80, CD83, CD205, MHC I, MHC II. The remaining cells were then resuspended at 1×10⁶ cells/ml with DC media containing 100 μg/ml of tumor lysate and supplemented with GM-CSF. Cells were then plated on T225 or T125 tissue culture flasks and incubated at 37° C. for 20 to 24 hours.

A TLR agonist and IFNγ were then added to each well, if appropriate as specified in Table 1, for 3 hours, at which point the appropriate inflammation-activating lipid was added directly to the culture medium, at a concentration of 120 μM. Flasks were rocked gently to mix and incubated at 37° C. for another 16 to 20 hours. Non-adherent and loosely adherent cells were harvested by gently rinsing with culture medium. The cell suspension was placed on ice. Ice cold DPBS was then added to the flask, and any remaining cells were incubated at 2 to 8° C. for 30 minutes. Cells adhering to the plates were gently scraped with a cell scraper. Cells were collected and combined with non-adherent fractions. Cells were rinsed twice with HBSS. Wash buffer was aspirated to remove additional buffer. Cells were again analyzed by flow cytometry, as before. Remaining cells were resuspended in Cryostar® freeze media (Biolife) at a cell density of 5×10⁶ cells per ml. Cells were cryopreserved in cryopreservation medium with 10% DSMO (CryoStor) for later use in preparation for dosing.

TABLE 1
Stimulation Conditions, Systemic Administration
of Tumor Lysate-Pulsed Hyperactivated DCs

| Treatment Group | Stimulation Conditions |
|---|---|
| 1 | DCs without treatment. (Negative control) |
| 2 | DCs + Tumor Lysate (Positive Control) |
| 3 | DCs + LPS + oxPAPC + Tumor Lysate |
| 4 | DCs + LPS + PGPC + Tumor Lysate |
| 5 | DCs + LPS + POV-PC + Tumor Lysate |
| 6 | DCs + LPS + PGPC + Tumor Lysate + IFNγ |
| 7 | DCs + LPS + POV-PC + Tumor Lysate + IFNγ |
| 8 | DCs + Poly I:C + oxPAPC + Tumor Lysate |
| 9 | DCs + Poly I:C + PGPC + Tumor Lysate |
| 10 | DCs + Poly I:C + POV-PC + Tumor Lysate |
| 11 | DCs + R848 + oxPAPC + Tumor Lysate |
| 12 | DCs + R848 + PGPC + Tumor Lysate |
| 13 | DCs + R848 + POV-PC + Tumor Lysate |
| 14 | DCs + LPS + Tumor Lysate |
| 15 | DCs + Poly I:C + Tumor Lysate |
| 16 | DCs + R848 + Tumor Lysate |

### Study Procedures

Mice were divided into four separate studies, with each study involving a different syngeneic mouse model cell line. At study start, mice were age-matched (8-12 weeks). Growth kinetics and expected take-rate influenced the number of tumor cells injected into the mice, with CT26 mice receiving a 0.1 ml injection of 3×10⁵ cells in Matrigel® and mice in the other three studies (i.e., 4T-1, A20, and Madison 109) receiving a 0.1 ml injection of 1×10⁶ tumor cells in Matrigel®. All tumor cell injections were administered subcutaneously to the right flank. Pair-matching was performed when tumors reached an average size of 80 to 120 mm³, at which point treatment began. Each study contained 16 groups of (up to) eight female BALB/c mice, where two of these groups were assigned as the positive and the negative control.

On days 1, 8, and 15, the cryopreserved stimulated DCs from each of the groups in Table 1 were thawed and resuspended in RPMI-1640 (without additives) at a target density of 1×10⁶ cells/50 μl for each individual therapeutic injection. Mice were injected intradermally in the left flank, opposite the flank where the tumor cells had been injected, with 3×10⁵ to 1×10⁶ cells (counts varied, depending on batch yields) mature activated DCs, at a volume of 0.05 ml/mouse. Body weight was recorded every day for the first 5 days, and then twice per week until study end. Tumors were measured with calipers twice per week until study end. Tumor Growth Delay (TGD) was the study endpoint, with the study ending when the tumor reached a pre-specified size (i.e., 1,000 mm³ in the 4T-1 model, and 2,000 mm³ in the CT-26, A20, and Madison 109 models) or when 45 days had passed, whichever occurred first.

### Discussion

These studies can show that groups of BALB/c mice treated therapeutically with BALB/c DCs that have been pulsed with syngeneic tumor cell lysate and matured with different stimulating conditions (i.e., TLR agonists and/or IFNγ and/or inflammation-activating lipids) will generate different outcomes with respect to reaching the study endpoint of tumor growth delay (TGD). Stimulating DCs with TLR agonists and inflammation-activating lipids should enable delivery of hyperactivated DCs to the injection site, where DCs will migrate to draining lymph nodes and secrete increased levels of pro-inflammatory cytokines (e.g., IL-1β) in situ for sustained periods of time, avoiding pyroptosis, and generating an enhanced T-cell mediated inflammatory response. T-cell responses should be strong enough to delay and/or reverse tumor growth in some instances and prolong life.

### Example 8: Intratumoral In Vivo Studies—CT26, 4T-1, Madison 109, A20 Syngeneic Tumor Cell Lines

In this example, BALB/c mouse DCs were stimulated ex vivo with various combinations of a TLR and IFN-γ, followed by activation with an inflammation-activating lipid, to create hyperactivated DCs. Tumors were measured for size to determine if the DCs could inhibit tumor cell proliferation and tumor growth.

As in Example 7, above, the same three TLR agonists (i.e., LPS, Poly J:C, and R848) and the same three inflammation-activating lipids (i.e., oxPAPC, PGPC, and POV-PC) were explored in this example. Tumors derived from the same four (4) syngeneic carcinoma mouse cell lines as selected above were initiated in BALB/c mice.

This example as compared to Example 7 above includes the use of a different mode of administration (intra tumoral versus systemic delivery), and the location/site of antigen uptake and processing by the DCs (in vivo versus ex vivo). In Example 7, DCs were pulsed with tumor lysate ex vivo, further stimulated with a TLR in combination with IFNγ, and an inflammation-activating lipid, and then injected systemically (intradermally). In this example, the DCs are naïve to tumor cells whilst ex vivo, where they are stimulated with a TLR in combination with IFNγ, and then activated with an inflammation-activating lipid. The DCs are collected and formulated for injection directly into a tumor mass in situ, where antigens are presented to DCs, taken up and processed, and wherein the DCs subsequently migrate to a draining lymph node to present antigen to naïve T cells to mount an antigen specific immune response.

In this example all four intratumoral studies were conducted with “fresh” BALB/c bone marrow-derived cells as the starting material for DC precursor cells. The cells in all four studies were prepared by Cellero. The DCs were only cryopreserved once, at the end of the cell stimulation process, following a 7-day DC culture and subsequent stimulation with TLRs and inflammation-activating lipids, in preparation for the various intratumoral injections.

Therapeutic intratumoral DC vaccination doses for three of the four studies in this example (i.e., with the A20, Madison 109, and 4T-1 cell lines) will begin when tumors have reached approximately 60 to 80 mm³. The starting tumor volume for the CT26 intratumoral administration study will be approximately 80-120 mm³.

### Methods

Mononuclear cells were isolated from BALB/c bone-marrow (Cellero) and cultured to generate immature DCs, as described above. Flow cytometric phenotypic analysis was performed on a sample of the immature DCs, looking at the following panel of markers: CD11b, CD11c, CD14, CD40, CD69, CD80, CD83, CD86, CD205, MHC-I, MHC-II. The remaining cells were resuspended at 1×10⁶/ml with DC media supplemented with GM-CSF and were added to T225 or T125 tissue culture flasks and incubated at 37° C. for 20 to 24 hours.

An equal volume of 2× cocktail of TLR agonist and IFN-γ were added, as applicable, in DC media supplemented with GM-CSF directly to the culture, and flasks were rocked gently to mix. Three (3) hours later, 120 μM of inflammation-activating lipid was added, as applicable, according to Table 2. Flasks were rocked gently to mix and incubated at 37° C. for another 16 to 20 hours. Non-adherent and loosely adherent cells were harvested by gently rinsing with culture medium, and cell suspension was placed on ice. Ice cold DPBS was added to flask and incubated at 2-8° C. for 30 minutes. Cells adhering to plates were gently scraped with a cell scraper, and cells were collected and combined with non-adherent fraction. Cells were rinsed twice with HBSS. Wash buffer was aspirated to remove additional buffer. Cells were analyzed by flow cytometry, as before. The remaining cells were resuspended in Cryostar® freeze media (Biolife) at a cell density of 5×10⁶ cells per ml. Cells were cryopreserved in cryopreservation medium with 10% DSMO (CryoStor) for later use in intratumoral administration studies.

TABLE 2
Stimulation Conditions, Intratumoral
Administration of Hyperactivated DCs

| Treatment Group | Stimulation Conditions |
|---|---|
| 1 | DCs without treatment. (Negative control) |
| 2 | DCs + R848 + IFNγ (Positive control) |
| 3 | DCs + LPS + oxPAPC |
| 4 | DCs + LPS + PGPC |
| 5 | DCs + LPS + POV-PC |
| 6 | DCs + LPS + PGPC + IFNγ |
| 7 | DCs + LPS + POV-PC + IFNγ |
| 8 | DCs + Poly I:C + oxPAPC |
| 9 | DCs + Poly I:C + PGPC |
| 10 | DCs + Poly I:C + POV-PC |
| 11 | DCs + R848 + oxPAPC |
| 12 | DCs + R848 + PGPC |
| 13 | DCs + R848 + POV-PC |
| 14 | DCs + LPS |
| 15 | DCs + Poly I:C |
| 16 | DCs + R848 |

R848 (1 μg/ml) and IFNγ (to 1,000 U/ml) were added to BALB/c DC cell suspension for 3 hours, after which point cells were plated in TC flasks and incubated for 16 to 20 hours. Adherent and non-adherent cells were harvested. The supernatant with the non-adherent cells was removed and the suspension placed on ice. Cold PBS was added to the wells, and the plate was placed in the refrigerator for 30 minutes to allow any remaining adherent cells to release. Cell scrapers were used on cells still sticking to the plates. The cell suspension with PBS was then collected and added to the previously harvested cells that have been kept on ice. The total collected cells were washed once with RPMI or HBSS prior to resuspension for injection.

Flow cytometry analysis was performed after stimulation, as above.

### Study Procedures

Mice were divided into four separate studies, with each study involving a different syngeneic mouse model cell line. At study start, mice were age-matched (8-12 weeks). Growth kinetics and expected take-rate influenced the number of tumor cells injected into the mice, with CT26 mice receiving 0.1 ml injections of 3×10⁵ cells in Matrigel® and mice in the other three studies (i.e., 4T-1, A20, and Madison 109 cell line studies) receiving 0.1 ml injections of 1×10⁶ tumor cells in Matrigel®. Pair-matching was performed among the mice to split them into treatment groups when their tumors reached an average size of 60 to 80 mm³ (for the A20, Madison 109, and 4T-1 cell line studies) or about 80 to 120 mm³ (for the CT26 study), at which point treatment began. Each study contained 16 groups, including a positive and negative control group, of (up to) eight female BALB/c mice.

DCs for each group of mice were thawed prior to injection, and at thaw, each individual group of DCs from Table Y was resuspended in RPMI-1640 (without additives) at a target density of 1×10⁶ cells/50 μl prior to injection. Mice received four 50 ml-per-dose DC vaccinations, on days 1, 2, 3, and 7. Mice were injected intratumorally with these DCs, according to the group-specific stimulation conditions in Table 2.

Body weight was recorded every day for the first 5 days, and then twice per week until study end. Tumors were measured with calipers twice per week until study end. Tumor Growth Delay (TGD) was the study endpoint, with the study ending when the tumor reached a pre-specified size (about 1,000 mm³ in the 4T-1 study; about 2,000 mm³ in the other three studies) or when 45 days had passed, whichever occurred first.

### Results

These studies show that groups of BALB/c mice treated therapeutically with BALB/c DCs that have been matured with different stimulating conditions (i.e., TLR agonists and/or IFNγ and/or inflammation-activating lipids) will generate different outcomes with respect to reaching the study endpoint of tumor growth delay (TGD). Stimulating DCs with TLR agonists and inflammation-activating lipids will hyperactivate DCs, and these DCs will pick up tumor-specific antigen in situ, later migrating to draining lymph nodes to pass antigen-specific messaging to T-cells. Upon injection and thereafter, these hyperactive, non-pyroptotic DCs will secrete increased levels of pro-inflammatory cytokines (e.g., IL-1β) in situ for longer than would otherwise be possible had they not been stimulated with inflammation-activating lipids. The inflammatory response may be sufficiently strong such that delay and/or reversal of tumor growth occurs in some instances, and mice remain alive longer as a result.

### Example 9: In Vitro or Ex Vivo Stimulation of Antigen Specific T-Cells

In this example, immature DC matured in the presence of the TLR agonist, in combination with IFNγ and the inflammation-activating lipid in the presence of tumor lysate are shown to stimulate enhanced levels of IFNγ production by antigen-specific T cells.

T cells are either isolated from, for example, peripheral blood, or can be stimulated in a cell population of blood constituents without isolation. The T cells are co-cultured with immature DCs, with DCs matured with a TLR agonist, such as LPS, BCG, Poly I:C, and/or R848, and IFNγ, or with DCs matured with a TLR agonist IFNγ, and an inflammation-activating lipid, such as, for example, oxPAPC. After an appropriate period of time the activated T cells can be isolated and assayed for the production of IFNγ using a commercially available ELISA assay.

The results will indicate that DCs stimulated with the TLR agonist, such as for example, LPS or BCG in combination with IFNγ and activated with the inflammation-activation lipid, such as, oxPAPC, are superior stimulators of antigen specific T cells. T cells co-cultured with immature DC are expected to produce very little IFNγ, while T cells co-cultured with DCs matured using BCG in combination with IFNγ would be expected to be intermediate producers of IFNγ. T cells co-cultured with DCs matured using the TLR agonist, such as BCG in combination with IFNγ and subsequently activated with the inflammation-activating lipid, such as oxPAPC, would be expected to produce the highest levels of IFNγ. Thus, DC matured with for example, BCG in combination with IFNγ and subsequent activation with oxPAPC are better stimulators of antigen-specific T cells.

The activated T cells produced by this method can be isolated and washed prior to formulating the T cells for administration to an individual. The T cells can also be cryopreserved for later use and thawed prior to administration.

### Example 10: Administration of Antigen Presenting Mature Dendritic Cells

Immature dendritic cells can be produced by any known method. For example, immature dendritic cells can be contacted in vitro or ex vivo with BCG and IFNγ as the maturation agent. Immature DCs can be prepared by contacting peripheral blood monocytes with plastic in the presence of OptiMEM® media (Gibco-BRL) supplemented with 1% human plasma. Unbound monocytes and other cells can be removed by washing. The bound monocytes can be cultured in, for example, X-VIVO 15® media in the presence of 500 U GM-CSF and 500 U IL-4 per milliliter for 6 days to form immature dendritic cells.

The immature dendritic cells can be subsequently cultured and matured with inactivated BCG in combination with IFNγ for a time period sufficient to fully mature the dendritic cells. During maturation the maturing dendritic cells are contacted with tumor lysate, for example, lysate from a glioma isolated from a patient to form tumor antigen presenting activated mature dendritic cells. The tumor antigen presenting activated mature dendritic cells can be either formulated for administration with or without the inflammation-activating lipid, for example oxPAPC. When formulated together with, for example, oxPAPC, the composition can be administered parenterally, for example, by systemic injection intravenously, intramuscularly, subcutaneously, or intratumorally into a tumor or surrounding tumor bed. When formulated as separate compositions, the composition comprising the tumor antigen presenting activated mature dendritic cells and the composition comprising the oxPAPC can be administered simultaneously or sequentially in any order. In one method the oxPAPC can be first applied topically to the area of the injection prior to administration of the composition comprising the tumor antigen presenting activated mature dendritic cells. For example, the composition comprising the oxPAPC can be applied topically to the area around the injection site for the dendritic cell composition.

### Example 11: Administration of Maturing Dendritic Cells with and without oxPAPC

In this example, maturing dendritic cells can be induced to mature with a combination of BCG, IFNγ, with or without oxPAPC. Prior to full maturation the dendritic cells, typically after 6 to 20 hours of culture, are isolated, washed and formulated for administration to an individual or prepared for cryopreservation in DMSO and human serum or plasma for later administration. When cryopreserved the composition is quickly thawed and if necessary diluted to the dosage for administration to the patient.

The patient when diagnosed with a solid tumor is treated with chemotherapy, radiation therapy, cryotherapy, or brachytherapy and subsequently administered intratumorally the maturing dendritic cell composition. Where the composition administered is the composition wherein the dendritic cells were induced to mature in the presence of oxPAPC, administration can be directly into the tumor or tumor bed surrounding the tumor. Where the composition comprising the maturing dendritic cells wherein oxPAPC was not used. The oxPAPC can be formulated either separately or together with the maturing dendritic cells. When formulated separately for administration to the subject the two compositions can be administered either simultaneously at the same or separate sites, or the compositions can be administered sequentially in either order. In one embodiment, where the oxPAPC and the maturing dendritic cells are formulated for separate administration, the oxPAPC is formulated for topical administration and is applied to the area of administration of the maturing dendritic cells prior to administration.

The previous examples are provided to illustrate, but not to limit, the scope of the claimed inventions. Other variants of the inventions will be readily apparent to those of ordinary skill in the art and encompassed by the appended claims. All publications, patents, patent applications and other references cited herein and are also incorporated by reference herein in their entirety.

While illustrative embodiments have been illustrated and described, it will be appreciated that various changes can be made therein without departing from the spirit and scope of the invention.

1. An in vitro or ex vivo method for producing a cell population enriched for hyperactive mature dendritic cells that produce an enhanced immune response to a predetermined antigen, the method comprising:
providing immature dendritic cells; and
contacting the immature dendritic cells with an effective amount of a dendritic cell maturation agent, with or without Interferon gamma (IFNγ), and an inflammation-activating lipid under culture conditions suitable for maturation of the immature dendritic cells for a time period sufficient to form mature dendritic cells;
wherein the mature dendritic cell population produces an enhanced Th1 response to the antigen when contacted with naïve T cells.

2. The method of claim 1, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

3. The method of claim 1, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

4. The method of any one of claims 1 to 3, wherein the immature dendritic cells are contacted with the dendritic cell maturation agent for at least about 3 hours prior to the addition of the inflammation-activating lipid, and the dendritic cells are cultured for an additional about 1 to 24 hours, or more.

5. The method of any one of claims 1 to 4, wherein the method further comprises contacting the maturing dendritic cells with a predetermined antigen prior to, simultaneously with, or subsequent contacting the immature dendritic cells with the dendritic cell maturation agent, with or without IFNγ, and the oxidized inflammation-activating lipid.

6. The method of claim 5, wherein the predetermined antigen is a tumor specific antigen, a tumor associated antigen, a viral antigen, a bacterial antigen, a tumor cell, a bacterial cell, a bacterial or tumor cell lysate, a bacterial or tumor cell membrane preparation, a recombinantly produced antigen, a peptide antigen, or an isolated antigen.

7. The method of claim 6, wherein the recombinantly produced antigen, peptide antigen or isolated antigen is a bacterial, a tumor associated, or a tumor specific antigen, peptide antigen, or isolated antigen.

8. The method of claim 1, further comprising:
obtaining a cell population enriched for monocytic dendritic cell precursors; and
culturing the precursors in the presence of a dendritic cell differentiating agent to form the immature dendritic cells.

9. The method of claim 8, wherein the dendritic cell differentiating agent is GM-CSF, or a combination of GM-CSF and Interleukin 4 (IL-4), Interleukin 7 (IL-7), Interleukin 13 (IL-13), or Interleukin 15 (IL-15).

10. The method of claim 8, wherein the monocytic dendritic cell precursors are isolated from a human subject.

11. The method of any one of claim 1 to 10, wherein the enhanced immune response is a Th1 response.

12. An in vitro or ex vivo method for producing an enhanced immune response to an antigen, the method comprising:
providing immature dendritic cells;
contacting the immature dendritic cells with an effective amount of a dendritic cell maturation agent, with or without Interferon gamma (IFNγ), and a predetermined antigen under culture conditions suitable for maturation of the immature dendritic cells to form the mature dendritic cell population;
wherein the maturing dendritic cells take up and process antigen during maturation;
formulating the mature dendritic cells for administration to an individual;
administering the mature dendritic cell formulation together with an effective amount of an inflammation-activating lipid to produce an enhanced Th1 immune response to the predetermined antigen as compared with the formulated dendritic cell composition administered without the inflammation-activating lipid.

13. The method of claim 12, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

14. The method of claim 12, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

15. The method of claim 12, wherein the predetermined antigen is a tumor specific antigen, a tumor associated antigen, a viral antigen, a bacterial antigen, a tumor cell, a bacterial cell, a bacterial or tumor cell lysate, a bacterial or tumor cell membrane preparation, a recombinantly produced antigen, a peptide antigen, or an isolated antigen.

16. The method of claim 15, wherein the recombinantly produced antigen, peptide antigen or isolated antigen is a bacterial, a tumor associated, or a tumor specific antigen, peptide antigen, or isolated antigen.

17. The method of claim 12, further comprising:
obtaining a cell population enriched for monocytic dendritic cell precursors; and
culturing the precursors in the presence of a dendritic cell differentiating agent to form the immature dendritic cells.

18. The method of claim 17, wherein the dendritic cell differentiating agent is GM-CSF, or a combination of GM-CSF and Interleukin 4 (IL-4), Interleukin 13 (IL-13), or Interleukin 15 (IL-15).

19. The method of claim 17, wherein the monocytic dendritic cell precursors are isolated from a human subject.

20. The method of any one of claims 12 to 19, wherein the formulated mature dendritic cells and the inflammation-activating lipid are administered simultaneously or sequentially in any order.

21. The method of any one of claims 12 to 20, wherein the enhanced immune response is a Th1 response.

22. An in vitro or ex vivo method for producing a hyperactivated mature dendritic cell population, comprising:
providing a cell population enriched for immature dendritic cells; and
contacting the immature dendritic cells with an effective amount of a dendritic cell maturation agent; with or without Interferon gamma (IFNγ); and an inflammation-activating lipid under culture conditions suitable for maturation of the immature dendritic cells to a time period sufficient to form a hyperactivated mature dendritic cell population.

23. The method of claim 22, further comprising contacting the immature dendritic cells with a predetermined antigen prior to or simultaneously contacting with the dendritic cell maturation agent; with or without Interferon gamma (IFNγ); or the inflammation-activating lipid.

24. The method of claim 22, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

25. The method of any one of claims 22 to 24, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

26. The method of any one of claims 22 to 25, wherein the method further comprises contacting the immature dendritic cells with a predetermined antigen.

27. The method of claim 26, wherein the predetermined antigen is a tumor specific antigen, a tumor associated antigen, a viral antigen, a bacterial antigen, a tumor cell, a bacterial cell, a recombinant cell expressing an antigen, wherein the antigen is a bacterial antigen, a viral antigen, a tumor associated antigen, or a tumor specific antigen, a bacterial or tumor cell lysate, a bacterial or tumor cell membrane preparation, a recombinantly produced antigen, a peptide antigen, or an isolated antigen, wherein the recombinantly produced antigen, peptide antigen or isolated antigen are bacterial, viral, tumor associated, or tumor specific antigens.

28. The method of claim 22, further comprising: isolating monocytic dendritic cell precursors; and culturing the precursors in the presence of a dendritic cell differentiation agent to form the immature dendritic cells.

29. The method of claim 28, wherein the differentiating agent is GM-CSF, or a combination of GM-CSF and IL-4, IL-13, or IL-15.

30. The method of claim 28, wherein the monocytic dendritic cell precursors are isolated from a human subject.

31. The method of any one of claims 22 to 30, wherein the activity of the mature dendritic cells is measured by an increase in a Th1 response, preferably wherein the increase in Th1 is measured by an increase in effector and memory T cell generation induced by the mature dendritic cells; an increase in terminal differentiation and/or activation of CD4⁺ an CD8⁺ T cells induced by the mature dendritic cells; and an increase in the secretion of IL1β, IL-2, and/or TNFα, by the mature dendritic cells.

32. Use of hyperactivated dendritic cells produced by any of the methods of claims 22 to 31, in making a medicament for the treatment of cancer, a bacterial infection, or a viral infection.

33. A composition for activating antigen specific T cells, comprising:
a cell population enriched for dendritic cells matured with an effective concentration of a dendritic cell maturation agent, with or without IFNγ, and an inflammation-activating lipid under culture conditions suitable for maturation of the immature dendritic cells and for a time period sufficient to form hyperactive mature dendritic cells; and
a predetermined antigen;
wherein the cell population enriched for hyperactive mature dendritic cells produces an enhanced T cell response as compared with mature dendritic cells matured with the effective concentration of a dendritic cell maturation agent; with or without IFNγ; and without the inflammation-activating lipid, preferably wherein the enhanced T cell response is a Th1 response.

34. The composition of claim 33, wherein the cell population enriched for hyperactive mature dendritic cells produces an enhanced antigen specific Th1 response when measured in vitro or in vivo and wherein the enhanced Th cell response is an increase in effector and memory T cell generation; an increase in terminal differentiation and/or activation of CD4⁺ and CD8⁺ T cells; and/or an increase in the secretion of IL1β, IL-2, and/or TNFα.

35. The composition of claim 33, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

36. The composition of claim 33, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

37. The composition of claim 33, wherein the predetermined antigen is a tumor specific antigen, a tumor associated antigen, a viral antigen, a bacterial antigen, a tumor cell, a bacterial cell, a recombinant cell expressing an antigen, a bacterial or tumor cell lysate, a bacterial or tumor cell membrane preparation, a recombinantly produced antigen, a peptide antigen, or an isolated antigen, wherein the recombinantly produced antigen, peptide antigen or isolated antigen are a bacterial, a viral, a tumor associated, or a tumor specific antigen.

38. An isolated, hyperactive mature dendritic cell population, comprising:
isolated hyperactive mature dendritic cells, produced by contacting immature dendritic cells with an effective concentration of a dendritic cell maturation agent; with or without IFNγ; and an inflammation-activating lipid under culture conditions suitable for maturation of the immature dendritic cells for a time period sufficient to form the hyperactive mature dendritic cells;
wherein the hyperactive mature dendritic cells produce an enhanced T cell response, preferably wherein the enhanced T cell response is an increase in effector and memory T cell generation when contacted with naïve T cells; an increase in terminal differentiation and/or activation of CD4⁺ and CD8⁺ T cells; and an increase in the secretion of IL1β.

39. The cell population of claim 38, further comprising a predetermined antigen.

40. The cell population of claim 38, further comprising isolated T cells.

41. The cell population of claim 40, wherein the T cells are naïve T cells.

42. The cell population of claim 38, further comprising isolated lymphocytes.

43. The cell population of claim 38, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly [C(12)U], or the TLR9 agonist is a sequence of nucleic acids containing unmethylated CpG motifs known to induce the maturation of DC, or any combination thereof.

44. The cell population of claim 38, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

45. An in vitro method for producing activated antigen specific T cells, comprising:
providing immature dendritic cells;
contacting the immature dendritic cells with a predetermined antigen;
contacting the immature dendritic cells with an effective concentration of a dendritic cell maturation agent; with or without IFNγ; and an inflammation-activating lipid under culture conditions suitable for maturation of the immature dendritic cells for a time period sufficient to form a hyperactive mature dendritic cell population; and
contacting the hyperactive mature dendritic cells with naïve T cells to form activated antigen specific T cells producing an increased amount of IFNγ as compared with T cells activated with a dendritic cell maturation agent with or without IFNγ, and without the inflammation-activating lipid.

46. The in vitro method of claim 45, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly [C(12)U], or the TLR9 agonist is a sequence of nucleic acids containing unmethylated CpG motifs known to induce the maturation of dendritic cells, or any combination thereof.

47. The in vitro method of claim 45, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

48. The method of claim 45, wherein the predetermined antigen is a tumor specific antigen, a tumor associated antigen, a viral antigen, a bacterial antigen, tumor cells, bacterial cells, recombinant cells expressing an antigen, wherein the antigen is a bacterial antigen, a viral antigen, a tumor associated antigen, or a tumor specific antigen; a cell lysate, wherein the cell lysate is a bacterial or a tumor cell lysate; a membrane preparation, wherein the membrane preparation is a bacterial or a tumor cell membrane preparation; a recombinantly produced antigen, a peptide antigen, or an isolated antigen, wherein the recombinantly produced antigen, the peptide antigen and the isolated antigen is a bacterial antigen, a viral antigen, a tumor associate antigen or a tumor specific antigen.

49. The method of claim 45, wherein the immature dendritic cells are contacted prior to or simultaneously with the predetermined antigen, and the dendritic cell maturation agent; with or without IFNγ; and the inflammation-activating lipid.

50. The method of claim 45, further comprising:
isolating monocytic dendritic cell precursors; and
culturing the precursors in the presence of a dendritic cell differentiation agent to form the immature dendritic cells.

51. The method of claim 50, wherein the dendritic cell differentiation agent is GM-CSF, or a combination of GM-CSF and IL-4, IL-7, IL-13, or IL-15.

52. The method of claim 50, wherein the monocytic dendritic cell precursors are isolated from a human subject.

53. The method of any one of claims 45 to 52, wherein the immature dendritic cells and T cells are autologous to each other.

54. A composition comprising isolated or enriched hyperactive mature dendritic cells and a pharmaceutically acceptable carrier, wherein the hyperactive mature dendritic cells produce more IL-1β, produce an increase in effector and memory T cells; and produce an increase in terminal differentiation and/or activation of CD4⁺ and CD8⁺ T cells than mature dendritic cells prepared by maturation of immature dendritic cells with a composition comprising effective concentrations of the dendritic cell maturation agent; with or without IFNγ; without the inflammation-activating lipid under conditions suitable for the maturation of the dendritic cells.

55. A method for producing an enhanced immune response in an animal,
providing immature dendritic cells;
contacting the immature dendritic cells with effective amounts of a dendritic cell maturation agent; with or without IFNγ; and an inflammation-activating lipid, and a predetermined antigen under culture conditions sufficient for maturation of the immature dendritic cells to form mature dendritic cells;
contacting the mature dendritic cells with naïve T cells to form activated T cells producing an increased amount of IFNγ and/or IL-1 as compared with T cells activated with a dendritic cell maturation agent and IFNγ without the inflammation-activating lipid; and
administering the hyperactivated T cells to the animal;
wherein the enhanced immune response is preferably a Th1 response.

56. The method of claim 55, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

57. The in vitro method of claim 55, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

58. The method of claim 55, wherein the predetermined antigen is a tumor specific antigen, a tumor associated antigen, a viral antigen, a bacterial antigen, tumor cells, bacterial cells, recombinant cells expressing an antigen, wherein the antigen is a bacterial antigen, a viral antigen, a tumor associated antigen, or a tumor specific antigen, a cell lysate, wherein the cell lysate is a bacterial cell lysate or a tumor cell lysate, a membrane preparation, wherein the membrane preparation is a tumor cell or bacterial cell membrane preparation, a recombinantly produced antigen, a peptide antigen or an isolated antigen, wherein the recombinantly produced antigen, peptide antigen and the isolated antigen have an epitope specific to or associated with a bacteria, a virus, or a tumor.

59. The method of claim 55, wherein the immature dendritic cells are contacted prior to or simultaneously with the predetermined antigen, the dendritic cell maturation agent; with or without IFNγ; and the inflammation-activating lipid.

60. The method of claim 55, further comprising:
isolating monocytic dendritic cell precursors from the animal; and culturing the precursors in the presence of a dendritic cell differentiation agent to form the immature dendritic cells.

61. The method of claim 60, wherein the dendritic cell differentiation agent is GM-CSF, or a combination of GM-CSF and IL-4, IL-13, or IL-15.

62. The method of claim 55, wherein the immature dendritic cells and T cells are autologous or allogeneic to the animal.

63. The method of claim 55, wherein the immature dendritic cells and T cells have the same MHC haplotype.

64. The method of claim 55, wherein the animal is human.

65. The method of claim 55, wherein the administration is parenteral.

66. The method of claim 65, wherein parenteral administration is intravenous, intradermal, subcutaneous, oral, transdermal, transmucosal, intratumoral, or rectal.

67. The method of claim 55, wherein the inflammation-activating lipid is applied to the site of administration.

68. An in vitro method for producing an anti-tumor immune response comprising:
providing immature dendritic cells;
contacting the immature dendritic cells with an effective amount of a dendritic cell maturation agent; with or without IFNγ; under culture conditions suitable for inducing maturation of the immature dendritic cells; and
isolating the maturing dendritic cells prior to full maturation;
formulating the maturing dendritic cells for administration;
administering the maturing dendritic cells intratumorally; and
administering an inflammation-activating lipid either intratumorally or systemically.

69. The method of claim 66, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

70. The in vitro method of claim 65, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

71. The method of claim 68, wherein the dendritic cells are obtained from skin, spleen, bone marrow, thymus, lymph node, umbilical cord blood, or peripheral blood.

72. The method of claim 68, wherein the dendritic cells are obtained from an individual to be treated or from a healthy individual HLA-matched to the individual to be treated.

73. A method for producing an anti-tumor immune response in an individual, the method comprising:
providing immature dendritic cells;
contacting the immature dendritic cells with a predetermined antigen, an effective amount of a dendritic cell maturation agent; with or without IFNγ; and an inflammation-activating lipid, under culture conditions suitable for inducing maturation of the immature dendritic cells and for a time period sufficient for the dendritic cells to uptake and process the predetermined antigen and to mature the dendritic cells;
isolating the mature dendritic cells;
formulating the isolated maturing dendritic cells for administration to the individual; and
administering the maturing dendritic cells intratumorally directly to the tumor.

74. The method of claim 73, wherein the dendritic cell maturation agent is a Toll-like Receptor (TLR) agonist, wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

75. The in vitro method of claim 73, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

76. The method of claim 73, wherein the dendritic cells are obtained from skin, spleen, bone marrow, thymus, lymph node, umbilical cord blood, or peripheral blood.

77. The method of claim 73, wherein the dendritic cells are obtained from the individual to be treated or from a healthy individual HLA-matched to the individual to be treated.

78. The method of any one of claims 73 to 77, wherein the mature dendritic cells are cryopreserved and are thawed prior to administration.

79. A method for producing an enhanced anti-tumor immune response in an individual, the method comprising:
providing immature dendritic cells;
contacting the immature dendritic cells with an effective amount of a dendritic cell maturation agent; with or without IFNγ under culture conditions suitable for inducing maturation of the immature dendritic cells;
isolating the maturing dendritic cells prior to full maturation;
formulating the isolated maturing dendritic cells for administration to the individual; and
co-administering the maturing dendritic cells to the individual with an inflammation-activating lipid.

80. The method of claim 79, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

81. The in vitro method of claim 76, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

82. The method of claim 79, wherein the dendritic cells are obtained from skin, spleen, bone marrow, thymus, lymph node, umbilical cord blood, or peripheral blood.

83. The method of claim 79, wherein the dendritic cells are obtained from the individual to be treated or from a healthy individual HLA-matched to the individual to be treated.

84. The method of claim 79, wherein the isolated maturing dendritic cells are cryopreserved and are thawed prior to administration.

85. The method of any one of claims 79 to 84, wherein the isolated maturing dendritic cells and the inflammation-activating lipid are administered simultaneously or sequentially in any order.

86. The method of claim 85, wherein the inflammation-activating lipid is in the same composition, or in separate compositions.

87. The method of claim 85, wherein the inflammation-activating lipid is in a separate composition and is applied topically to the site of administration.

88. An in vitro method for producing a mature dendritic cell population that produces an enhanced Th1 response to an antigen, the method comprising:
providing immature dendritic cells; and
contacting the immature dendritic cells with an effective amount of a dendritic cell maturation agent; with or without Interferon gamma (IFNγ); and an inflammation-activating lipid under culture conditions suitable for maturation of the immature dendritic cells for a sufficient time to form the mature dendritic cell population;
wherein the mature dendritic cell population produces an enhanced Th1 response to the antigen when contacted with naïve T cells.

89. The method of claim 88, wherein the dendritic cell maturation agent is a Toll-like Receptor (TLR) agonist, preferably wherein the TLR agonist is a TLR2 agonist, a TLR3 agonist, a TLR4 agonist, a TLR7 agonist, and/or a TLR8 agonist, or a TLR9, more preferably wherein the TLR4 agonist is LPS, or BCG, the TLR7 and/or TLR8 agonist is an imidazoquinoline compound, preferably, a imidazoquinoline-4-amine compound, more preferably 4-amino-2-ethoxymethyl-α,α-dimethyl-1H-imidazol[4,5-c]quinolin-1-ethanol (R848) or 1-(2-methylpropyl)-1H-imidazo[4,5-c]quinolin-4-amine (R837), and their derivatives, the TLR3 agonist is a synthetic double stranded polyribonucleotide, preferably poly I:C or poly [I]:poly[C(12)U], or the TLR9 agonist is a sequence of nucleic acids containing unmethylated CpG motifs known to induce the maturation of DC, or any combination thereof.

90. The method of claim 85, wherein the inflammation-activating lipid is one or more of oxidized 1-palmitoyl-2-arachidonyl-sn-glycero-3-phosphorylcholine (oxPAPC), 1-palmitoyl-2-(5-oxovaleroyl)-sn-glycero-3-phosphatidylcholine (POV-PC), 1-palmitoyl-2-glutaroyl-sn-glycero-3-phosphatidylcholine (PGPC) and 1-palmitoyl-2-(5,6-epoxyisopropane E₂)-sn-glycero-3-phosphatidylcholine (PEIPC), 1-palmitoyl-2-(5-hydroxy-8-oxo-6-octenedioyl) sn-glycero-3-phosphocholine (HOdiA-PC), 1-palmitoyl-2-(5-keto-6-octene-dioyl)-sn-glycero-3-phosphocholine (KOdiA-PC), 1-palmitoyl-2-(5-hydroxy-8-oxooct-6-enoyl)-sn-glycero-3-phosphocholine (HOOA-PC), 1-palmitoyl-(5-keto-8-oxo-6-octenoyl)-sn-glycero-3-phosphocholine (KOOA-PC).

91. The method of any one of claims 88 to 90, wherein the method further comprising contacting the immature dendritic cells with a predetermined antigen.

92. The method of claim 91, wherein the predetermined antigen is a tumor specific antigen, a tumor associated antigen, a viral antigen, a bacterial antigen, a tumor cell, a bacterial cell, a bacterial or tumor cell lysate, a bacterial or tumor cell membrane preparation, a recombinantly produced antigen, a peptide antigen, or an isolated antigen.

93. The method of claim 92, wherein the recombinantly produced antigen, peptide antigen or isolated antigen is a bacterial, a tumor associated, or a tumor specific antigen, peptide antigen, or isolated antigen.

94. The method of claim 88, further comprising contacting the immature dendritic cells with a predetermined antigen prior to or simultaneously with contacting the immature dendritic cells with the dendritic cell maturation agent, with or without IFNγ, and the oxidized inflammation-activating lipid.

95. The method of claim 88, further comprising:
isolating monocytic dendritic cell precursors; and
culturing the precursors in the presence of a dendritic cell differentiating agent to form the immature dendritic cells.

96. The method of claim 95, wherein the dendritic cell differentiating agent is GM-CSF, or a combination of GM-CSF and Interleukin 4 (IL-4), Interleukin 13 (IL-13), or Interleukin 15 (IL-15).

97. The method of claim 95, wherein the monocytic dendritic cell precursors are isolated from a human subject.
